(19) United States
(12) Patent Application Publication
(10) Pub. No.: US 2026/0287575 A1
(43) Pub. Date: Sep. 24, 2026

(54) LABEL-FREE METHODS FOR ASSESSING CELL STATE IN A BIOLOGICAL SAMPLE

(71) Applicant: Elephas Biosciences Corporation, Madison, WI (US)

(72) Inventors: Jason Smith, Madison, WI (US); Chao Liu, Madison, WI (US); Jonathan Ouellette, Madison, WI (US); Jonathan Daniel Oliner, Garrett Park, MD (US); Eric Wait, Madison, WI (US); John Daniel Rafter, Madison, WI (US)

(21) Appl. No.: 19/475,618

(22) PCT Filed: Apr. 17, 2024
(86) PCT No.: PCT/US2024/025016

§ 371 Date: Oct. 15, 2025

Related U.S. Application Data
(60) Provisional application No. 63/459,804, filed on Apr. 17, 2023.

(51) Int. Cl. G01N 33/50; G01N 15/01; G01N 15/1433; G01N 21/64; G02B 21/16

(52) U.S. Cl. CPC G01N 33/505; G01N 15/01; G01N 15/1433; G01N 21/6458; G01N 33/502; G02B 21/16

(57) ABSTRACT

Provided herein are methods of assessing cell state within a biological sample. In particular, the present invention provides techniques for identifying a live cell fraction within a biological sample through use of OCM/dOCM imaging, and determining a metabolic signature of the identified live cell fraction through use of FLIM imaging. The present invention further provides techniques for assessing changes in cell state of a live cell fraction within a biological sample through use of OCM/dOCM and FLIM imaging in response to a pharmaceutical intervention.

The present application claims priority to U.S. Provisional Application No. 63/459,804, filed Apr. 17, 2023, which is incorporated herein by reference in its entirety.

### FIELD OF THE INVENTION

Provided herein are methods of assessing cell state within a biological sample. In particular, the present invention provides techniques for identifying a live cell fraction within a biological sample through use of OCM/dOCM imaging, and determining a metabolic signature of the identified live cell fraction through use of FLIM imaging. The present invention further provides techniques for assessing changes in cell state of a live cell fraction within a biological sample through use of OCM/dOCM and FLIM imaging in response to a pharmaceutical intervention.

### BACKGROUND

Label-free determination of cell viability (such as live, necrotic, apoptotic, and necroptotic etc.) within a living biological sample (e.g., a living tissue sample) has high potential in several applications including ex vivo drug response assessment. Scanning multi-photon fluorescence microscopy (MPM) assesses cells within a sample using near infrared (NIR) light to penetrate into tissue and produce three dimensional images. However, the ability of MPM to differentiate cell state is limited in samples with high heterogeneity, such as tumor tissue samples.

Accordingly, what is needed are improved methods for accurate assessment of cell state in a heterogenous tissue sample.

The present invention addresses these needs.

### SUMMARY

Experiments described herein (see, Examples) demonstrated an ability to contemporaneously or nearly-contemporaneously 1) identify a live cell fraction and/or a necrotic cell fraction within a biological sample through use of optical coherence microscopy (OCM) imaging and/or dynamic optical coherence microscopy (dOCM) imaging, and 2) determine a baseline metabolic signature for the identified live cell fraction of the biological sample through use of fluorescence lifetime imaging microscopy (FLIM).

Accordingly, provided herein are methods of assessing cell state within a biological sample. In particular, the present invention provides techniques for identifying a live cell fraction within a biological sample (e.g., living tissue sample, organs, or bodily fluids such as whole blood, plasma, serum, tissue, lavage or any other specimen) (e.g., living tissue sample from whole biopsies or biopsies that have been cut longitudinally into one or more strips) (e.g., living tissue sample comprising one or more of a whole biopsy, a bisected biopsy tissue sample, and a living tissue fragment(s)) (e.g., living tissue sample including “tissue coins” cut from a tissue biopsy) through use of OCM/dOCM imaging, and determining a metabolic signature of the identified live cell fraction through use of FLIM. The present invention further provides techniques for assessing changes in cell state of a live cell fraction within a biological sample through use of OCM/dOCM and FLIM imaging in response to a pharmaceutical intervention.

In certain embodiments, the present invention provides methods of evaluating a biological sample, comprising a) imaging the biological sample with OCM/dOCM (e.g., OCM and/or dOCM) to identify a live cell fraction in the biological sample; and b) imaging the biological sample with FLIM to classify the cell state within the identified live cell fraction.

In some embodiments, imaging the biological sample with FLIM comprises imaging the live cell fraction identified with OCM/dOCM.

In some aspects, the step of OCM/dOCM occurs prior to the step of FLIM. In some aspects, the step of FLIM occurs prior to the step of OCM/dOCM. In some aspects, the OCM/dOCM and FLIM steps occur contemporaneously or nearly contemporaneously. In some aspects, each assessment is contemporaneous or nearly-contemporaneous.

In some aspects, the imaging the biological sample with OCM/dOCM to identify a live cell fraction in the biological sample and the imaging the biological sample with FLIM to classify the cell state is contemporaneous or nearly contemporaneous. In some aspects, the biological sample is one or more selected from a living tissue sample, an organ sample, a whole blood sample, a plasma sample, a serum sample, and a lavage sample. In some aspects, the living tissue sample is selected from one or more of: whole biopsies or biopsies that have been cut longitudinally into one or more strips; tissue coins cut from a tissue biopsy; a whole biopsy; a bisected biopsy tissue sample, and a living tissue fragment. In some aspects, the biological sample comprises cytotoxic T-cell lymphocytes (CTLs).

In some aspects, classifying the cell state with FLIM comprises classifying the metabolic state and/or the metabolic signature in the identified live cell fraction. In some aspects, the FLIM comprises multi-photon FLIM (MP-FLIM). In some aspects, MP-FLIM provides a signal from a first intrinsic fluorophore and a signal from a second intrinsic fluorophore, wherein the first intrinsic fluorophore is NAD(P)H and/or the second intrinsic fluorophore is FAD. In some aspects, classifying cell state comprises calculating a lifetime metabolic ratio (LMR) of the identified live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD. In some aspects, the signal from the first intrinsic fluorophore and the signal from the second intrinsic fluorophore are identified by phasor segmentation analysis. In some aspects, the signal from the first intrinsic fluorophore and the signal from the second intrinsic fluorophore are identified by phasor segmentation analysis. In some aspects, the signal from the first intrinsic fluorophore corresponds to an amount of free NAD(P)H and wherein the signal from the second intrinsic fluorophore corresponds to an amount of enzyme-bound FAD. In some aspects, an increased signal from the first intrinsic fluorophore relative to a threshold value and/or an increased signal from the second intrinsic fluorophore relative to a threshold value indicates a glycolytic metabolic state in the biological sample.

In some aspects, the biological sample is further evaluated with one or more of the following additional imaging modalities: mass spectrometry (MS), nuclear magnetic resonance spectroscopy, light sheet microscopy, lattice light sheet microscopy, magnetic resonance imaging, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography), SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonics generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

In some aspects, the methods further comprise: c) contacting the biological sample with one or more potential therapeutic agents, wherein the contacting is subsequent to the imaging of the biological sample with OCM/dOCM and FLIM; and d) imaging the biological sample with OCM/dOCM and/or FLIM to re-classify the cell state within the identified live cell fraction to measure classification differences pre- and post-contacting of the biological sample with the one or more potential therapeutic agents.

In some aspects, measuring the classification differences comprises measuring a metabolic shift of the identified and classified live cell fraction from a baseline metabolic state. In some aspects, the baseline metabolic state is determined prior to contacting the identified and classified live cell fraction with the potential therapeutic agent.

In some aspects, the one or more potential therapeutic agents is selected from one or more of a small molecule, a polypeptide or peptide fragment, an siRNA, a cellular therapy (e.g., tumor-infiltrating lymphocyte therapy, CAR T-cell therapy, T-cell receptor-based therapy, natural killer cell therapy, etc.), an oncolytic virus, a metabolite treatment (e.g., an adenosine treatment), or an antibody or fragment thereof.

In certain embodiments, the present invention provides ex vivo methods of assessing one or more potential therapeutic agents, comprising a) imaging a biological sample with OCM/dOCM to identify a live cell fraction in the biological sample; b) imaging the biological sample with FLIM to classify the cell state within the biological; c) contacting the biological sample with one or more potential therapeutic agents, wherein the contacting is subsequent to the imaging of the biological sample with OCM/dOCM and FLIM; and d) imaging the biological sample with OCM/dOCM and/or FLIM to re-classify the cell state within the biological sample to measure classification differences pre- and post-contacting of the biological sample with the one or more potential therapeutic agents.

In some embodiments, imaging the biological sample with FLIM comprises imaging the live cell fraction identified with OCM/dOCM.

In some aspects, the step of OCM/dOCM occurs prior to the step of FLIM. In some aspects, the step of FLIM occurs prior to the step of OCM/dOCM. In some aspects, the OCM/dOCM and FLIM steps occur contemporaneously or nearly contemporaneously. In some embodiments, each assessment is contemporaneous or nearly-contemporaneous.

In some aspects, the biological sample is one or more selected from a living tissue sample, an organ sample, a whole blood sample, a plasma sample, a serum sample, and a lavage sample. In some aspects, the living tissue sample is selected from one or more of: whole biopsies or biopsies that have been cut longitudinally into one or more strips; tissue coins cut from a tissue biopsy; a whole biopsy; a bisected biopsy tissue sample; and a living tissue fragment. In some aspects, the biological sample comprises CTLs.

In some aspects, classifying the cell state with FLIM comprises classifying the metabolic state. In some aspects, the FLIM comprises multi-photon FLIM (MP-FLIM). In some aspects, MP-FLIM provides a signal from a first intrinsic fluorophore and a signal from a second intrinsic fluorophore, wherein the first intrinsic fluorophore is NAD(P)H and/or the second intrinsic fluorophore is FAD. In some aspects, the signal from the first intrinsic fluorophore and the signal from the second intrinsic fluorophore are identified by phasor segmentation analysis. In some aspects, the signal from the first intrinsic fluorophore corresponds to an amount of free NAD(P)H and wherein the signal from the second intrinsic fluorophore corresponds to an amount of enzyme-bound FAD.

In some aspects, the assessment differences pre- and post-contacting of the biological sample with the one or more potential therapeutic agents comprises measuring a metabolic shift of the biological sample from a baseline metabolic state. In some aspects, the baseline metabolic state is determined prior to contacting the biological sample with the potential therapeutic agent.

In some aspects, the one or more potential therapeutic agents is selected from one or more of a small molecule, a polypeptide or peptide fragment, an siRNA, a cellular therapy, an oncolytic virus, a metabolite treatment (e.g., an adenosine treatment), or an antibody or fragment thereof. In some aspects, the cellular therapy is one or more selected from tumor-infiltrating lymphocyte therapy, CAR T-cell therapy, T-cell receptor-based therapy, and natural killer cell therapy.

In certain embodiments, the present invention provides ex vivo methods of assessing a biological sample, comprising providing a biological sample, and a processor comprising software that, when executed, causes the processor to manually or automatically: image the biological sample with OCM and/or dOCM to identify a live cell fraction in the biological sample; and image the biological sample with FLIM to classify the cell state within the identified live cell fraction; and provide a report to a user of the identified and classified live cell fraction;
executing the processor to image the biological sample with OCM/dOCM to identify a live cell fraction in the biological sample; and image the biological sample with FLIM to classify the cell state within the identified live cell fraction, and provide a report to a user of the identified and classified live cell fraction.

In some aspects, the imaging the biological sample with OCM and/or dOCM to identify a live cell fraction in the biological sample and the imaging the biological sample with FLIM to classify the cell state within the biological sample is contemporaneous or nearly contemporaneous. In some aspects, the biological sample is one or more selected from a living tissue sample, an organ sample, a whole blood sample, a plasma sample, a serum sample, and a lavage sample. In some aspects, the living tissue sample is selected from one or more of: whole biopsies or biopsies that have been cut longitudinally into one or more strips; tissue coins cut from a tissue biopsy; a whole biopsy; a bisected biopsy tissue sample, and a living tissue fragment. In some aspects, the biological sample comprises CTLs.

In some aspects, classifying the cell state with FLIM comprises classifying the metabolic state in the biological sample. In some aspects, the FLIM comprises multi-photon FLIM (MP-FLIM). In some aspects, MP-FLIM provides a signal from a first intrinsic fluorophore and a signal from a second intrinsic fluorophore, wherein the first intrinsic fluorophore is NAD(P)H and/or the second intrinsic fluorophore is FAD. In some aspects, the signal from the first intrinsic fluorophore and the signal from the second intrinsic fluorophore are identified by phasor segmentation analysis. In some aspects, the signal from the first intrinsic fluorophore and the signal from the second intrinsic fluorophore are identified by phasor segmentation analysis. In some aspects, the signal from the first intrinsic fluorophore corresponds to an amount of free NAD(P)H and wherein the signal from the second intrinsic fluorophore corresponds to an amount of enzyme-bound FAD. In some aspects, an increased signal from the first intrinsic fluorophore relative to a threshold value and/or an increased signal from the second intrinsic fluorophore relative to a threshold value indicates a glycolytic metabolic state in the biological sample. In some aspects, classifying cell state comprises calculating a lifetime metabolic ratio (LMR) of the biological sample, wherein the LMR is calculated by dividing a signal from the first intrinsic fluorophore corresponding to the amount of enzyme-bound NAD by a signal from the second intrinsic fluorophore corresponding to the amount of enzyme-bound FAD.

In some aspects, the biological sample is further evaluated with one or more of the following additional imaging modalities: mass spectrometry (MS), nuclear magnetic resonance spectroscopy, magnetic resonance imaging, light sheet microscopy, lattice light sheet microscopy, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography), SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonics generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

In some aspects, the processor further comprises a display configured to display the provided report.

In certain embodiments, the present invention provides ex vivo methods of assessing one or more potential therapeutic agents, comprising providing: a biological sample, and a processor comprising software that, when executed, causes the processor to manually or automatically: image the biological sample with OCM/dOCM to identify a live cell fraction in the biological sample; image the biological sample with FLIM to classify the cell state within the biological sample; contact the biological sample with one or more potential therapeutic agents subsequent to the imaging of the biological sample with OCM/dOCM and FLIM; image the biological sample with OCM/dOCM and/or FLIM to re-classify the cell state within the biological sample; measure classification differences pre- and post-contacting of the biological sample with the one or more potential therapeutic agents; provide a report to a user of one or more of: an identified and classified live cell fraction; a re-classified identified and classified live cell fraction; and classification differences pre- and post-contacting of the biological sample with the one or more potential therapeutic agents; executing the software to: image the biological sample with OCM/dOCM to identify a live cell fraction in the biological sample; image the biological sample with FLIM to classify the cell state within the biological sample; contact the biological sample with one or more potential therapeutic agents subsequent to the imaging of the biological sample with OCM/dOCM and FLIM; image the biological sample with OCM/dOCM and/or FLIM to re-classify the cell state within the biological sample; measure classification differences pre- and post-contacting of the biological sample with the one or more potential therapeutic agents; provide a report to a user of one or more of: an identified and classified live cell fraction; a re-classified identified and classified live cell fraction; and classification differences pre- and post-contacting of the biological sample with the one or more potential therapeutic agents.

In some aspects, the imaging the biological sample with OCM/dOCM (e.g., OCM and/or dOCM) to identify a live cell fraction in the biological sample and the imaging the biological sample with FLIM to classify the cell state within the identified live cell fraction is contemporaneous or nearly contemporaneous.

In some aspects, the biological sample is one or more selected from: a living tissue sample, an organ sample, a whole blood sample, a plasma sample, a serum sample, and a lavage sample. In some aspects, the living tissue sample is selected from one or more of: whole biopsies or biopsies that have been cut longitudinally into one or more strips; tissue coins cut from a tissue biopsy; a whole biopsy; a bisected biopsy tissue sample, and a living tissue fragment.

In some aspects, the one or more potential therapeutic agents is selected from one or more of a small molecule, a polypeptide or peptide fragment, an siRNA, a cellular therapy (e.g., tumor-infiltrating lymphocyte therapy, CAR T-cell therapy, T-cell receptor-based therapy, natural killer cell therapy, etc.), an oncolytic virus, a metabolite treatment (e.g., an adenosine treatment), or an antibody or fragment thereof.

In some aspects, classifying the cell state with FLIM comprises classifying the metabolic state in the biological sample. In some aspects, the FLIM comprises multi-photon FLIM (MP-FLIM). In some aspects, MP-FLIM provides a signal from a first intrinsic fluorophore and a signal from a second intrinsic fluorophore, wherein the first intrinsic fluorophore is NAD(P)H and/or the second intrinsic fluorophore is FAD. In some aspects, the signal from the first intrinsic fluorophore and the signal from the second intrinsic fluorophore are identified by phasor segmentation analysis. In some aspects, the signal from the first intrinsic fluorophore and the signal from the second intrinsic fluorophore are identified by phasor segmentation analysis. In some aspects, the signal from the first intrinsic fluorophore corresponds to an amount of free NAD(P)H and wherein the signal from the second intrinsic fluorophore corresponds to an amount of enzyme-bound FAD. In some aspects, an increased signal from the first intrinsic fluorophore relative to a threshold value and/or an increased signal from the second intrinsic fluorophore relative to a threshold value indicates a glycolytic metabolic state in the biological sample. In some aspects, classifying cell state comprises calculating a lifetime metabolic ratio (LMR) of the biological sample, wherein the LMR is calculated by dividing a signal from the first intrinsic fluorophore corresponding to the amount of enzyme-bound NAD by a signal from the second intrinsic fluorophore corresponding to the amount of enzyme-bound FAD.

In some aspects, the biological sample is further evaluated with one or more of the following additional imaging modalities: mass spectrometry (MS), nuclear magnetic resonance spectroscopy, magnetic resonance imaging, light sheet microscopy, lattice light sheet microscopy, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography). SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonics generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

In some aspects, the processor further comprises a display configured to display the provided report.

In certain embodiments, the present invention provides a system comprising: a biological sample, and a processor comprising software that, when executed, causes the processor to manually or automatically: image the biological sample with OCM/dOCM (e.g., OCM and/or dOCM) to identify a live cell fraction in the biological sample; and image the biological sample with FLIM to classify the cell state within the biological sample; and provide a report to a user of the identified and classified live cell fraction and/or biological sample.

In certain embodiments, the present invention provides a system comprising: a biological sample, and a processor comprising software that, when executed, causes the processor to manually or automatically: image the biological sample with OCM/dOCM (e.g., OCM and/or dOCM) to identify a live cell fraction in the biological sample; image the biological sample with FLIM to classify the cell state within the biological sample; contact the biological sample with one or more potential therapeutic agents subsequent to the imaging of the biological sample with OCM/dOCM and FLIM; image the biological sample with OCM/dOCM and/or FLIM to re-classify the cell state within the biological sample; measure classification differences pre- and post-contacting of the biological sample with the one or more potential therapeutic agents; provide a report to a user of one or more of an identified and classified live cell fraction; a re-classified identified and classified live cell fraction; classification differences pre- and post-contacting of the biological sample with the one or more potential therapeutic agents; and classification differences pre- and post-contacting of identified and classified live cell fraction with the one or more potential therapeutic agents.

In certain embodiments, the present invention provides ex vivo methods of assessing one or more potential therapeutic agents, comprising a) imaging a biological sample with OCM and/or dOCM to identify a live cell fraction in the biological sample; b) imaging the identified live cell fraction with FLIM to classify the cell state within the identified live cell fraction; c) contacting the identified and classified live cell fraction with one or more potential therapeutic agents; and d) imaging the identified live cell fraction with OCM/dOCM and/or FLIM to re-classify the cell state within the identified live cell fraction to measure classification differences pre- and post-contacting of the identified and classified live cell fraction with the one or more potential therapeutic agents.

In certain embodiments, the present invention provides an ex vivo method of assessing a biological sample, comprising providing a biological sample, and a processor comprising software that, when executed, causes the processor to manually or automatically: image the biological sample with OCM and/or dOCM to identify a live cell fraction in the biological sample; and image the identified live cell fraction with FLIM to classify the cell state within the identified live cell fraction; and provide a report to a user of the identified and classified live cell fraction; executing the processor to image the biological sample with OCM and/or dOCM to identify a live cell fraction in the biological sample; and image the identified live cell fraction with OCM/dOCM and/or FLIM to classify the cell state within the identified live cell fraction, and provide a report to a user of the identified and classified live cell fraction.

In certain embodiments, the present invention provides an ex vivo method of assessing one or more potential therapeutic agents, comprising providing a biological sample, and a processor comprising software that, when executed, causes the processor to manually or automatically: image the biological sample with OCM and/or dOCM to identify a live cell fraction in the biological sample; image the identified live cell fraction with FLIM to classify the cell state within the identified live cell fraction; contact an identified and classified live cell fraction with one or more potential therapeutic agents after an identification and classification of a live cell fraction with OCM and/or dOCM and FLIM; image the identified and classified live cell fraction with FLIM to re-classify the cell state within the identified and classified live cell fraction; measure classification differences pre- and post-contacting of identified and classified live cell fraction with the one or more potential therapeutic agents; provide a report to a user of one or more of: an identified and classified live cell fraction; a re-classified identified and classified live cell fraction; and classification differences pre- and post-contacting of identified and classified live cell fraction with the one or more potential therapeutic agents; executing the software to: image the biological sample with optical coherence microscopy (OCM) and/or dynamic optical coherence microscopy (dOCM) to identify a live cell fraction in the biological sample; image the identified live cell fraction with FLIM to classify the cell state within the identified live cell fraction; contact an identified and classified live cell fraction with one or more potential therapeutic agents after an identification and classification of a live cell fraction with OCM and/or dOCM and FLIM; image the identified and classified live cell fraction with fluorescence lifetime imaging microscopy (FLIM) to re-classify the cell state within the identified and classified live cell fraction; measure classification differences pre- and post-contacting of identified and classified live cell fraction with the one or more potential therapeutic agents; provide a report to a user of one or more of: an identified and classified live cell fraction; a re-classified identified and classified live cell fraction; and classification differences pre- and post-contacting of identified and classified live cell fraction with the one or more potential therapeutic agents.

In certain embodiments, the present invention provides a system comprising: a biological sample, and a processor comprising software that, when executed, causes the processor to manually or automatically: image the biological sample with OCM/dOCM to identify a live cell fraction in the biological sample; and image the identified live cell fraction with FLIM to classify the cell state within the identified live cell fraction; and provide a report to a user of the identified and classified live cell fraction.

In certain embodiments, the present invention provides a system comprising a biological sample, and a processor comprising software that, when executed, causes the processor to manually or automatically: image the biological sample with OCM/dOCM to identify a live cell fraction in the biological sample; image the identified live cell fraction with FLIM to classify the cell state within the identified live cell fraction; contact an identified and classified live cell fraction with one or more potential therapeutic agents after an identification and classification of a live cell fraction with OCM and/or dOCM and FLIM; re-image the identified and classified live cell fraction with OCM/dOCM and/or FLIM to re-classify the cell state within the identified and classified live cell fraction; measure classification differences pre- and post-contacting of identified and classified live cell fraction with the one or more potential therapeutic agents; provide a report to a user of one or more of: an identified and classified live cell fraction; a re-classified identified and classified live cell fraction; and classification differences pre- and post-contacting of identified and classified live cell fraction with the one or more potential therapeutic agents.

In some aspects, provided herein are methods of evaluating a biological sample. In some embodiments, provided herein is a method of evaluating a biological sample, comprising imaging a biological sample with optical coherence microscopy (OCM) and/or dynamic optical coherence microscopy (dOCM), thereby identifying a live cell fraction in the biological sample, and imaging the biological sample with multi-channel fluorescence lifetime imaging microscopy (FLIM) to classify cell state within the live cell fraction of the biological sample. In some embodiments, FLIM comprises multi-channel FLIM. In some embodiments, multi-channel FLIM provides a signal from a first intrinsic fluorophore and a signal from a second intrinsic fluorophore. In some embodiments, the first intrinsic fluorophore is NAD(P)H and the second intrinsic fluorophore is FAD. In some embodiments, the signal from the first intrinsic fluorophore and the signal from the second intrinsic fluorophore are identified by phasor segmentation analysis.

In some embodiments, classifying cell state comprises calculating a lifetime metabolic ratio (LMR) of the live cell fraction. In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD.

In some embodiments, provided herein is a method of evaluating a biological sample, comprising imaging a biological sample with optical coherence microscopy (OCM) and/or dynamic optical coherence microscopy (dOCM), thereby identifying a live cell fraction of the biological sample, and imaging the biological sample with fluorescence lifetime imaging microscopy (FLIM) to determine a baseline metabolic signature for the live cell fraction of the biological sample. In some embodiments, FLIM comprises multi-channel FLIM. In some embodiments, multi-channel FLIM provides a signal from a first intrinsic fluorophore and a signal from a second intrinsic fluorophore. In some embodiments, the first intrinsic fluorophore is NAD(P)H and the second intrinsic fluorophore is FAD. In some embodiments, the signal from the first intrinsic fluorophore and the signal from the second intrinsic fluorophore are identified by phasor segmentation analysis.

In some embodiments, the baseline metabolic signature is determined by calculating a lifetime metabolic ratio (LMR) of the live cell fraction. In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD.

In some aspects, provided herein are methods of evaluating a potential therapeutic agent. In some embodiments, provided herein is a method of evaluating a potential therapeutic agent, comprising imaging a biological sample with optical coherence microscopy (OCM) and/or dynamic optical coherence microscopy (dOCM), thereby identifying a live cell fraction of a biological sample, contacting the biological sample with a potential therapeutic agent, and measuring a metabolic shift in the live cell fraction from a baseline metabolic signature for the live cell fraction. In some embodiments, the method further comprises classifying cell state within the live cell fraction based upon the metabolic shift measured in the live cell fraction.

In some embodiments, the baseline metabolic signature for the live cell fraction is determined by imaging the biological sample with fluorescence lifetime imaging microscopy (FLIM). In some embodiments, FLIM comprises multi-channel FLIM. In some embodiments, multi-channel FLIM provides a signal from a first intrinsic fluorophore and a signal from a second intrinsic fluorophore. In some embodiments, the first intrinsic fluorophore is NAD(P)H and the second intrinsic fluorophore is FAD. In some embodiments, the signal from the first intrinsic fluorophore and the signal from the second intrinsic fluorophore are identified by phasor segmentation analysis.

In some embodiments, the baseline metabolic signature is determined by calculating a lifetime metabolic ratio (LMR) of the live cell fraction. In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD.

In some embodiments, the metabolic shift in the live cell fraction is measured by FLIM. In some embodiments, the metabolic shift in the live cell fraction is measured by multi-channel FLIM. In some embodiments, multi-channel FLIM provides a signal from a first intrinsic fluorophore and a signal from a second intrinsic fluorophore. In some embodiments, the first intrinsic fluorophore is NAD(P)H and the second intrinsic fluorophore is FAD.

In some embodiments, the metabolic shift in the live cell fraction is determined by calculating a lifetime metabolic ratio (LMR) of the live cell fraction. In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD. In some embodiments, the signal from the first intrinsic fluorophore and the signal from the second intrinsic fluorophore are identified by phasor segmentation analysis.

In some embodiments, a metabolic shift above a threshold value is indicative of cell death. In some embodiments, a metabolic shift within a first range of reference values is indicative of apoptosis, and/or a metabolic shift within a second range of reference values is indicative of necrosis. For example, in some embodiments, a metabolic shift within a first range of reference values is indicative of apoptosis. As another example, in some embodiments a metabolic shift within a second range of reference values is indicative of necrosis.

In some embodiments, the potential therapeutic agent comprises a cell. In some embodiments, the cell comprises a leukocyte. For example, in some embodiments the cell comprises T-cell, a B-cell, an NK cell, or a macrophage. In some embodiments, the potential therapeutic agent comprises a drug. For example, in some embodiments the drug comprises a potential anti-cancer agent.

In some aspects, provided herein are methods of evaluating a biological sample, comprising contacting a biological sample with a leukocyte, and imaging the biological sample with fluorescence lifetime imaging microscopy (FLIM) to classify cell state in the biological sample. In some embodiments, FLIM comprises multi-channel FLIM.

In some embodiments, multi-channel FLIM provides a signal from a first intrinsic fluorophore and a signal from a second intrinsic fluorophore, wherein the first intrinsic fluorophore is NAD(P)H and the second intrinsic fluorophore is FAD. In some embodiments, the signal from the first intrinsic fluorophore and the signal from the second intrinsic fluorophore are identified by phasor segmentation analysis. In some embodiments, classifying cell state comprises calculating a lifetime metabolic ratio (LMR) of the live cell fraction. In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD. In some embodiments, the leukocyte comprises a T-cell, a B-cell, an NK cell, or a macrophage. Such cells may be used as potential therapeutic agents for the treatment of cancer. The anti-cancer activity of such cells can be evaluated using the methods described herein.

In some aspects, provided herein are methods of evaluating the therapeutic potential of a leukocyte. In some embodiments, the method comprises contacting a biological sample with a leukocyte, measuring a metabolic shift in the biological sample from a baseline metabolic signature for the biological sample, and classifying cell state within the biological sample based upon the metabolic shift measured. In some embodiments, the baseline metabolic signature is determined by imaging the sample with FLIM. In some embodiments, FLIM comprises multi-channel FLIM. In some embodiments, multi-channel FLIM provides a signal from a first intrinsic fluorophore and a signal from a second intrinsic fluorophore, wherein the first intrinsic fluorophore is NAD(P)H and the second intrinsic fluorophore is FAD. In some embodiments, the signal from the first intrinsic fluorophore and the signal from the second intrinsic fluorophore are identified by phasor segmentation analysis. In some embodiments, the baseline metabolic signature is determined by calculating a lifetime metabolic ratio (LMR). In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD.

In some embodiments, the metabolic shift is measured by FLIM. In some embodiments, the metabolic shift is measured by multi-channel FLIM. In some embodiments, multi-channel FLIM provides a signal from a first intrinsic fluorophore and a signal from a second intrinsic fluorophore. In some embodiments, the first intrinsic fluorophore is NAD(P)H and the second intrinsic fluorophore is FAD. In some embodiments, the metabolic shift is determined by calculating a lifetime metabolic ratio (LMR), wherein the LMR is calculated by dividing a signal from the first intrinsic fluorophore corresponding to the amount of enzyme-bound NAD by a signal from the second intrinsic fluorophore corresponding to the amount of enzyme-bound FAD. In some embodiments, the signal from the first intrinsic fluorophore and the signal from the second intrinsic fluorophore are identified by phasor segmentation analysis.

In some embodiments, a metabolic shift above a threshold value is indicative of cell death. In some embodiments, a metabolic shift within a first range of reference values is indicative of apoptosis, and/or a metabolic shift within a second range of reference values is indicative of necrosis.

For any of the embodiments described herein, the biological sample may comprise a tissue sample. In some embodiments, the biological sample comprises a tumor tissue sample.

### BRIEF DESCRIPTION OF THE DRAWINGS

FIG. 1 demonstrates the capability of dOCM to distinguish live and dead cells. A 300 μm thick slice of liver is shown. Local injection of 200 nL staurosporine (STS, 2 μM in DMSO) caused tissue death in the upper part of the xy plane. Live cells in the live region (lower part in the xy plane) are highlighted in the dOCM image volume. The live cells show high contrast compared to dead regions in the three orthogonal views (xy, yz, and xz planes). The positions of the yz and xz slices are indicated by the yellow lines in the xy plane. Scale bar: 100 μm.

FIG. 2 shows a comparison of propidium iodide (PI) staining imaged by two-photon microscopy to dOCM images in the same tissue. PT is a fluorescent intercalating agent, which is not permeant to live cells. Only nuclei in the dead cells are stained by PI. As shown in FIG. 2A, dOCM shows a much higher contrast between live and dead tissues compared to PI staining. The percentage of live tissue in the total area of the tissue slice is calculated as 7% by applying threshing to the d(OCM image as shown in FIG. 2B. The two images are averaged projections of 50 μm in depth.

FIG. 3 shows a comparison of the lifetime metabolic ratio (LMR) obtained by FLIM to dOCM imaging in another mouse liver slice. As shown in FIG. 3A, tissue necrosis occurs on edges of a tissue obtained by biopsy, thus providing a natural boundary of live (center) and dead (edges) cells within the tissue sample. A biopsy needle was used to obtain the 1 mm tissue sample and caused tissue necrosis in the edges of the mouse liver. As shown in FIG. 3B, cellular structures were visualized in the center of the tissue but not in the dead edges. In the LMR image, the live region shows much lower values compared to the dead regions. The two images are averaged projections of 50 μm in depth. Scale bar: 100 μm.

FIGS. 4A-4B show an overview of exemplary methods described herein. As shown in FIG. 4A, left panel, dOCM can be used to obtain respective images of live and dead cell fractions. As shown in FIG. 4A, right panel, the LMR for the live cell fraction (confirmed by dOCM) and the dead cell fraction (confirmed by dOCM) can be determined. Exemplary probability density function graphs of live and dead cell fractions are shown in FIG. 4B, with the graph on the left corresponding to the probability density function based upon dOCM imaging and the graph on the right corresponding to the probability density function based upon the LMR calculated for live and dead cell fractions, respectively. Some sections of this image, highlighted by regions labelled by red dashed lines in FIG. 4A, are not classified properly by the LMR alone. The dOCM is used to establish a ground truth for living tissue that can in turn be used to qualify the LMR and set a baseline value for living tissue in these regions. Scale bars: 100 μm.

FIG. 4C-D shows multimodal imaging of tissue viability with a piece of mouse liver using dOCM and FLIM. 100 nL shikonin (6 μM) was injected into the center of the tissue to create necroptosis indicted by the white arrow. Scale bar: 100 μm. FIG. 4C shows composite image of dOCM indicating viable cells (green) and dead tissue stained by propidium iodide (magenta). FIG. 4D shows lifetime metabolic ratio (LMR) measured by FLIM from the same tissue. LMR values are indicated by the color bar. Viable cells exhibit higher LMR values compared to dead cells.

FIG. 5 shows that the methods described herein can be used to evaluate cell state in a primary human oral cancer sample. The gray scale image shows dOCM imaging of a tissue sample, which accurately identifies the live cell fraction (and, correspondingly, the dead cell fraction) of the sample. The image on the top right shows the LMR for the same tissue sample, corroborating the live and dead cell fractions visualized using dOCM. The image on the bottom right shows PI (for dead cells) and Caspase 3/7 (for apoptotic cells) staining, which further identifies portions of the tissue undergoing cell death. These imaging modalities can be used in combination via the methods described herein to identify cell state in a biological sample with a high degree of accuracy and specificity. Scale bars: 100 μm.

FIG. 6 shows applying LMR alone cannot classify live and dead cells. A CT26 live tumor fragment was treated with 3-MA, a selective autophagy and phosphoinositide 3-kinase (PI3K) inhibitor, and stained with PI. PI staining indicates dead cells as magenta in the lower panel. However, these cells show relatively uniform low LMR values.

FIG. 7 shows the process by which cells were segmented for downstream analysis using the lifetime metabolic ratio relative to ground truth dyes. Here, a CT26 live tumor fragment (300×300×300 microns) was imaged (FLIM) using an 8 channel Swabian instruments time tagger at 740 nm and 880 nm. The resultant FLIM data were analyzed as described to determine the LMR. A. Data derived from the intensity signals were used to segment the image into individual Voronoi cells seeded from nuclear segmentation. The signal from propidium iodide, which indicates cell death is overlayed on the image. B. LMR histogram data from the fragments was normalized by taking the mode of the resultant histogram data at baseline from healthy tissue and setting this value to 0 across the time course. The normalized LMR value was determined for each pixel and plotted. C. Ground truth for each Voronoi cell was determined using the dye propidium iodide and used as a classifier for each Voronoi cell to determine shifts in the LMR relative to baseline indicating a cell had died.

FIGS. 8A-8C show application of the LMR and ground truth dyes in tissue that underwent induced modes of cell death via staurosporine (apoptosis), Shikonin (necroptosis), or heat shock (another mode of apoptosis). The ground truth sensor probe for caspase 3 or 7 (FIG. 8A) and PI (FIG. 8B) was used to distinguish apoptotic modalities from necroptotic modalities. The resulting normalized LMR shifts were plotted in time relative to time zero baseline and two threshold shifts were determined indicative of either apoptosis of necroptosis (FIG. 8C). Area under the receiver operating characteristic (AUROC) logistic regression (10-fold cross-validation) analysis on data presented in the confusion matrices was used to determine the shift values with highest statistical significance for each mode of cell death.

FIG. 9 demonstrates the use of LMR shift values determined in CT26 syngeneic control data sets on a different syngeneic mouse line, MCA205. Experiments on MCA205 LTFs were performed as described for CT26 LTF (FIG. 8) and statistical analysis performed on the resulting qualified data as described in the text.

FIG. 10 shows the application of LMR shift values to follow the time course of a combination of meta-methoxyamphetamine (3MA) and Doxorubicin administered to CT26 LTFs. Decline in cell viability as determined in cells that had exceeded threshold shift values were verified using propidium iodide. Statistical analysis showed good correlation (AUC value of 0.92) for the 48 hour time point.

FIG. 11 describes the generation and validation of TIL populations used in experiments described herein. TILs were isolated from CT26 fragments using negative selection for T cells. Briefly, CT26 fragments were incubated for 24 h to allow the efflux of TILs into the surrounding media. After the incubation period, the fragments were filtered from the media and the remaining cells in the media were subjected to negative selection for T cells following the standard protocol from Stem Cell Technologies. Flow cytometry and lactate dehydrogenase (LDH) release assays were performed on the TIL-CT26 co-cultures to verify T cell cytotoxicity. To measure CT26 cell death, the percent of dead CD45− cells was quantified using flow cytometry.

FIGS. 12A-B shows the use of phasor space analysis on FLIM data and the shifts in metabolic state for cells which come into contact with a targeted T cell. Briefly, TILs were isolated from excised murine CT26 tumors, subsequently activated and co-cultured with CT26 cells in vitro. FLIM images are shown in FIG. 12A. Analysis (e.g. phasor analysis) is shown in FIG. 12B. The normalized LMR shift was effective in identifying loss of viability in targeted CT26 cells. FIG. 12B also shows data from a single CT26 cell in contact with a T cell (identified by the bounding box and red arrow in 12A). This is in contrast to an adjacent CT26 cell not contacted by a T cell which shows no metabolic shift.

FIG. 13 shows the use of phasor space analysis on FLIM data taken in CT26 LTFs after addition of activated TILs. Whole tissue analysis shows a shift in the overall phasor space plot. Further inspection identified punctate regions where pronounced changes in LMR were observed, tying the shift in the overall phasor space plot to a small population of cells undergoing cell death and loss of viability.

FIG. 14 shows decreasing statistical accuracy of the use of LMR shifts in more complex tissues.

FIG. 15A-C present schematics of constructed microscope embodiments allowing for simultaneous visualization of a sample by OCM/dOCM and multi-channel multi-photon microscopy. FIG. 15A shows a system configuration that can employ OCM/dOCM from the top of the sample while either sequentially or simultaneously employ multi-photon imaging from the bottom of the sample. Also depicted are the scanners for the multiphoton, the time tagger for FLIM and the optics for separating out signals. The addition of the Swabian time tagger allows for multi-channel FLIM measurements described in this document. The configuration shown in FIG. 15B brings the multi-photon scanner in from one side of the microscope (right side depicted here) and the OCM/dOCM scanner from the other side of the microscope. This configuration is suitable for sequential imaging of the sample with OCM/dOCM and multi-photon imaging, FLIM. FIG. 15C shows a configuration which mounts the multi-photon scanner for imaging and FLIM from the right side of the microscope and the OCM/dOCM scanner from the bottom of the microscope. This configuration is suitable for either simultaneous multi-photon imaging and FLIM with OCM/dOCM or sequential imaging of the acquisition modes.

FIG. 16 presents multimodal imaging of tissue viability with primary human lung cancer tissue using combined dOCM and FLIM. (a) dOCM and LMR images of a human lung cancer tissue slice. (b) dOCM and LMR images of another lung tissue slice from the same sample. The metabolic state of this sample is different from the tissue slice in (a). (c) The tissue slice in (a) was treated with 10 μM staurosporine. The slice was imaged 24 hours after treatment. Less viable cells were revealed by dOCM; the metabolic state was shifted compared to that prior to treatment. (d) The shift of metabolism was visualized by the NADH and FAD phasor plots indicating sample undergoing apoptosis, consistent with FIG. 8C. Scale bar: 100 μm.

FIG. 17 presents multimodal imaging of primary human ovary cancer tissue with low viability using combined dOCM and FLIM. Live cells are revealed by the dOCM. The live cells showed different metabolic states (LMR) compared with the dead cells. Moreover, the metabolic states were different from the sample in FIG. 16 without treatment. Scale bar: 100 μm.

### DETAILED DESCRIPTION

### 1. Definitions

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

The phrase “and/or,” as used herein in the specification and in the claims, should be understood to mean “either or both” of the elements so conjoined, e.g., elements that are conjunctively present in some cases and disjunctively present in other cases.

As used herein, the term “OCM/dOCM” refers to OCM and/or dOCM as used herein in the specification and in the claims, should be understood to mean “either or both” of the elements so conjoined.

As used herein, the terms “assess”, “evaluate”, and “characterize” are used interchangeably.

The term “subject” as used herein is any mammalian or non-mammalian subject. The subject may be a primate or a non-primate subject. In some embodiments, the subject is suspected of or diagnosed with cancer. In some embodiments, the subject is a human subject. The cancer can be any solid or hematologic malignancy. The cancer can be of any stage and/or grade. Non-limiting examples of cancer include cancers of head & neck, oral cavity, breast, ovary, uterus, gastro-intestinal, colorectal, pancreatic, prostate, brain and central nervous system, skin, thyroid, kidney, bladder, lung, liver, bone and other tissues.

The term “biological sample” as used herein refers to any cellular biological material such as a tissue, a tissue fragment, cellular aggregates such as a spheroid or an organoid and the like. In preferred embodiments, the biological sample is a live sample (e.g., a live tissue sample) comprising viable cells. In some embodiments, a live biological sample is one which has not been subjected to any tissue fixation techniques (such as formalin fixation). In some embodiments, the biological sample is obtained from a subject. In some embodiments, the biological sample comprises healthy (e.g., not diseased) biological material such as a healthy tissue, a healthy tissue fragment, healthy cellular aggregates such as a spheroid or an organoid and the like.

While a tissue can be from any organ or site in the body of a subject, in some embodiments, the tissue (or tissue sample, as used interchangeably herein) contains or is suspected of containing tumor cells (also referred to as a tumor containing tissue or a tumor tissue sample). The terms tumor cells, cancerous cells, and malignant cells are used interchangeably herein. In some embodiments, the tissue is a solid tumor tissue. The tissue can be obtained from any organ or site in the body of the subject where a cancer has originated or where the cancer has metastasized to.

In some embodiments, the tissue (or tissue sample, as used interchangeably herein) contains healthy cells (e.g., not cancerous cells) (e.g., not diseased cells). In some embodiments, the tissue (or tissue sample, as used interchangeably herein) contains a combination of healthy cells (e.g., not cancerous cells) (e.g., not diseased cells) and non-healthy cells (e.g., cancerous cells) (e.g., diseased cells).

A tissue can be obtained from a subject by any approach known to a person skilled in the art. The tissue can be obtained by surgical resection, surgical biopsy, investigational biopsy, bone marrow aspiration or any other therapeutic or diagnostic procedure performed on a subject suspected of or diagnosed with cancer.

In some embodiments, the biological sample is an ex vivo living tissue sample that typically results from a biopsy. In some embodiments, the ex vivo living tissue sample is a tumor tissue sample. The term “tumor tissue sample” means any tumor tissue sample derived from the patient. In some embodiments, the tumor sample may result from the tumor resected from the patient. In some embodiments, the tumor sample may result from a biopsy performed in the primary tumor of the patient or performed in a metastatic sample distant from the primary tumor of the patient. In some embodiments, the tumor tissue sample encompasses (i) a global primary tumor (as a whole), (ii) a tissue sample from the centre of the tumor, (iii) lymphoid islets in close proximity with the tumor, (iv) the lymph nodes located at the closest proximity of the tumor, (v) a tumor tissue sample collected prior to surgery (for follow-up of patients after treatment for example), and (vi) a distant metastasis. In some embodiments, the tumor tissue sample, encompasses pieces or slices of tissue that have been removed from the tumor, including following a surgical tumor resection or following the collection of a tissue sample for biopsy. The tumor tissue sample can, of course, be subjected to a variety of well-known post-collection preparative and storage techniques. Typically, the cells are dissociated from the tissue in order to prepare a suspension of cells. A common method to obtain suspensions from primary tissue is enzymatic disaggregation.

In some embodiments, a live tissue or a live tissue fragment is one in which the cells are viable. In some embodiments, a live tissue or a live tissue fragment is one in which the viability of cells is not significantly altered compared to tissue that is freshly excised from the subject. In some embodiments, a live tissue or a live tissue fragment is one which has not been subjected to any tissue fixation techniques (such as formalin fixation). According to some embodiments, viable cells are cells with an intact cell membrane. According to some embodiments, the cell membranes of viable cells are largely impermeable to certain viability test molecules—such as propidium iodide (PI), 7-AAD and the like.

In preferred embodiments, the biological sample is an ex vivo live sample (e.g., a live tissue sample) or an ex vivo living tissue sample comprising viable cells. In some embodiments, a live tissue sample includes, but is not limited to, an ex vivo tissue sample, an ex vivo healthy tissue sample, an ex vivo biopsy sample, an ex vivo tissue resection, etc. In some embodiments, the live tissue sample contains a mixture of living and dead cells and/or tissue. In some embodiments, the live tissue sample contains a mixture of healthy and non-healthy cells and/or tissue. In some embodiments, the biological sample is obtained from a subject (e.g., a human subject).

Tissue fragments are obtained by cutting a tissue obtained from a subject. In some embodiments, the size of each tissue fragment is equal to or less than 1000 μm (such as 1000 μm, 500 μm, 450 μm, 400 μm, 350 μm, 300 μm, 250 μm, 200 μm, 100 μm or 50 μm) in at least one dimension. In some embodiments, the size of each tissue fragment is between 50 μm and 1000 μm in least one dimension. In some embodiments, the size of each tissue fragment is between 100 μm and 500 μm in at least one dimension. In some embodiments, the size of each tissue fragment is between 150 μm and 350 μm in at least one dimension. In some embodiments, the size of each tissue fragment is between 50 μm and 1000 μm in at least two dimensions. In some embodiments, the size of each tissue fragment is between 150 μm and 350 μm in two dimensions. In some embodiments, the size of each tissue fragment is between 50 μm and 500 μm in all three dimensions. In some embodiments, the size of each tissue fragment is between 150 μm and 350 μm in all three dimensions. In some embodiments, the size of each tissue fragment is about 300 μm in any two dimensions and about 100 μm in the third dimension. In some embodiments, the size of each tissue fragment is about 300 μm in all three dimensions. In some embodiments, the tissue fragments are substantially cubical in shape. In some embodiments, the tissue fragments are uniform in size. As used herein, uniform means substantially uniform wherein the size of the tissue fragments are within ±20% of one another, in at least one dimension.

As used herein, the term “cell” or “cells” refers to any eukaryotic cell. Eukaryotic cells include without limitation ovary cells, epithelial cells, immune cells, hematopoietic cells, bone marrow cells, circulating vascular progenitor cells, cardiac cells, chondrocytes, bone cells, beta cells, hepatocytes, and neurons. Moreover the term includes pluripotent stem cells. Such cells can be ex vivo cells, in vivo cells, in vitro, etc. In some embodiments, the population of cells consists of a homogeneous population of cells. As used herein, the term “homogeneous population of cells” refers to a population of cells comprising one cell type and/or one cell state. In some embodiments, the population of cells consists of a heterogeneous population of cells. As used herein, the term “heterogeneous population of cells” refers to a population of cells comprising two or more different cell types or cell states.

In some embodiments, the population of cells comprises immune cells. As used herein, the term “immune cell” includes cells that are of haematopoietic origin and that play a role in the immune response. Immune cells include lymphocytes, such as B cells and T cells; natural killer cells; myeloid cells, such as monocytes, neutrophils, macrophages, dendritic cells, eosinophils, mast cells, basophils, and granulocytes (see, also, Hu, et al., Molecular Biology Reports, 2022 49:9783-9795; herein incorporated by reference in its entirety).

In some embodiments, the population of cells comprises T cells. As used herein, the term “T cell,” refers to a type of lymphocyte that plays an important role in cell-mediated immunity and are distinguished from other lymphocytes, such as B cells, by the presence of a T-cell receptor on the cell surface. Several subsets of T cells have been described and typically include helper T cells (e.g., Thl, Th2, Th9 and Th17 cells), cytotoxic T cells, memory T cells, regulatory/suppressor T cells (Treg cells), natural killer T cells, [gamma/delta]T cells, and/or autoaggressive T cells (e g., TH40 cells), unless otherwise indicated by context. In some embodiments, the term “T cell” refers specifically to a helper T cell. In some embodiments, the term “T cell” refers more specifically to a TH17 cell (i.e., a T cell that secretes IL-17). In some embodiments, the term “T cell” refers to a regulatory T cell or “Treg” cell.

As used herein, the terms “exogenous label” or an “exogenous agent” are used interchangeably and refer to any external agent that enhances the contrast during imaging. A skilled artisan can envisage multiple types of exogenous labels depending on the modality of imaging, non-limiting examples of which can include an optical label (e.g., a fluorescent label), a magnetic label, an acoustic label and the like. In some embodiments, the exogenous label is a fluorescent label. An exogenous label is one that is added to a tissue fragment. In contrast, an “endogenous label” or an “intrinsic label” are used interchangeably herein and refer to a naturally occurring molecule in the tissue fragment or originating from the tissue fragment. An endogenous label is an intrinsic, naturally occurring molecule within the biological sample, which enhances the contrast while imaging the tissue fragments. In some embodiments, the endogenous label is an intrinsic fluorescent molecule (called an intrinsic fluorophore) present within the biological sample (such as the tissue or the tissue fragment). An intrinsic fluorophore is a naturally occurring fluorescent molecule in the biological sample. Non-limiting examples of intrinsic fluorophores include NADH (reduced form of nicotinamide adenine dinucleotide), NADPH (reduced form of nicotinamide adenine dinucleotide phosphate), flavins and flavin derivatives (such as flavin adenine dinucleotide (FAD)), aromatic amino acids such as tryptophan, tyrosine and the like. In some embodiments, the intrinsic fluorophore comprises one or more fluorescence lifetime components distinguished by fluorescence lifetimes.

“Fluorescence parameters” as used herein refers to one or more of fluorescence intensity (e.g., photon counts), fluorescence lifetimes parameters (e.g., individual lifetimes of the fluorescence lifetime components, mean lifetime etc.) or fluorescence lifetime composition (e.g., relative amplitudes of the fluorescence lifetime components etc.) of one or more fluorophores (such as intrinsic fluorophores). In some embodiments a set of fluorescence parameters comprises, photon counts from the intrinsic fluorophore, a weighted mean (rm) of the lifetimes of a plurality of fluorescence lifetime components of the intrinsic fluorophore and the amplitude (an) of at least one fluorescence lifetime component relative to the plurality of fluorescence lifetime components. In some embodiments, the set of fluorescence parameters further comprises individual lifetimes of at least two fluorescence lifetime components of the plurality of fluorescence lifetime components. In some embodiments lifetime components may derive from one or more spectral channels. In some embodiments one or more lifetime components may be derived from each spectral range of 420-500 nm and 501 nm-560 nm and 561 nm-660 nm.

As used herein, the term “processor” (e.g., a microprocessor, a microcontroller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor is a microprocessor that can be configured to communicate in a stand-alone and/or a distributed environment and can be configured to communicate via wired or wireless communications with other processors, where such one or more processor(s) can be configured to operate on one or more processor-controlled devices that can be similar or different devices.

As used herein, the term “memory” is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processor can be connected to the memory and execute software instructions that are capable of being stored in a RAM of the memory (e.g., during execution), a ROM of the memory (e.g., on a generally permanent bases), or another non-transitory computer readable medium such as another memory or a disc. In some embodiments, the memory includes one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor can be configured to retrieve from the memory and execute, among other things, instructions related to the processes and methods described herein.

As used herein, the term “network” generally refers to any suitable electronic network including, but not limited to, a wide area network (“WAN”) (e.g., a TCP/IP based network), a local area network (“LAN”), a neighbourhood area network (“NAN”), a home area network (“HAN”), or personal area network (“PAN”) employing any of a variety of communications protocols, such as Wi-Fi, Bluetooth, ZigBee, etc. In some embodiments, the network is a cellular network, such as, for example, a Global System for Mobile Communications (“GSM”) network, a General Packet Radio Service (“GPRS”) network, an Evolution-Data Optimized (“EV-DO”) network, an Enhanced Data Rates for GSM Evolution (“EDGE”) network, a 3GSM network, a 4GSM network, a 5G New Radio, a Digital Enhanced Cordless Telecommunications (“DECT”) network, a digital AMPS (“IS-136/TDMA”) network, or an Integrated Digital Enhanced Network (“iDEN”) network, etc. In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet).

### 2. Methods

Experiments described herein (see, Examples) demonstrated an ability to contemporaneously or nearly-contemporaneously 1) identify a live cell fraction and/or a necrotic cell fraction within a biological sample through use of optical coherence microscopy (OCM) imaging and/or dynamic optical coherence microscopy (dOCM) imaging, and 2) determine a baseline metabolic signature for the identified live cell fraction of the biological sample through use of fluorescence lifetime imaging microscopy (FLIM).

Accordingly, provided herein are methods of assessing cell state within a biological sample. In particular, the present invention provides techniques for identifying a live cell fraction within a biological sample through use of OCM/dOCM imaging, and determining a metabolic signature of the identified live cell fraction through use of FLIM imaging. The present invention further provides techniques for assessing changes in cell state of a live cell fraction within a biological sample through use of OCM/dOCM and FLIM imaging in response to a pharmaceutical intervention.

In some aspects, provided herein are methods.

In some embodiments, provided herein are methods of assessing and/or characterizing and/or evaluating a biological sample (e.g., living tissue sample, organs, or bodily fluids such as whole blood, plasma, serum, tissue, lavage or any other specimen) (e.g., living tissue sample from whole biopsies or biopsies that have been cut longitudinally into one or more strips) (e.g., living tissue sample comprising one or more of a whole biopsy, a bisected biopsy tissue sample, and a living tissue fragment(s)) (e.g., living tissue sample including “tissue coins” cut from a tissue biopsy) (e.g., healthy living tissue) (e.g., living tissue suspected of being healthy) (e.g., living tissue suspected of not being healthy). In some embodiments, provided herein are methods of assessing and/or characterizing and/or evaluating a potential therapeutic agent.

In some embodiments, the methods described herein involve imaging a biological sample with multiple imaging modalities to classify cell state within the biological sample. In some embodiments, the use of multiple imaging modalities facilitates identification of cell state within a heterogenous sample containing multiple cell types and/or cells of varying metabolic states, such as a tumor tissue sample. In some aspects, the methods of assessing and/or characterizing and/or evaluating a biological sample comprise contacting the biological sample with a potential therapeutic agent, and using one or multiple imaging modalities to classify cell state in the sample before and after contact with the potential therapeutic agent (e.g., to characterize and/or detect changes in the cell state before and after exposure to the potential therapeutic agent). Accordingly, the methods provided herein can be used to evaluate whether a potential therapeutic agent induces cell death (e.g. apoptosis, necroptosis) or other metabolic shifts towards cell death in the sample.

Such methods are not limited to assessment of a particular type of biological sample. In some embodiments, the biological sample is one or more of living tissue samples, organs, and bodily fluids such as whole blood, plasma, serum, tissue, lavage or any other specimen.

Such methods are not limited to assessment of a particular type of a living tissue sample. In some embodiments, the live tissue sample is a live tumor fragment culture. In some embodiments, the live tissue sample is a mixture of different types of living cells. In some embodiments, the live tissue sample comprises fragments of living tumor tissue, referred to herein as live tumor fragments or LTFs. In some embodiments, a live tissue sample includes, but is not limited to, an ex vivo tissue sample, an ex vivo biopsy sample, an ex vivo tissue resection, etc. In some embodiments, the living tissue sample is from whole biopsies or biopsies that have been cut longitudinally into one or more strips. In some embodiments, the living tissue sample includes “tissue coins” cut from a tissue biopsy.

In some embodiments, the living tissue sample includes immune cells. The term “immune cell” as used herein refers to lymphocytes (such as B cells and T cells); natural killer cells; myeloid cells (such as monocytes, macrophages and dendritic cells), neutrophils, eosinophils, mast cells, basophils, or granulocytes. The term “immune cell” is inclusive of “tumor infiltrating” immune cells. The term “tumor infiltrating” refers to an immune cell that is located inside a tumor. The term “tumor infiltrating immune cell” is inclusive of “tumor infiltrating lymphocytes”, which refer to tumor infiltrating T-cells, B-cells, and natural killer (NK) cells. For example, the method may comprise evaluating T-cells (e.g., activated T-cells, CD8+ T-cells, CD4+ T cells) B-cells, and/or NK cells. In some embodiments, the methods involve detecting the activity (e.g., motility) of T-cells (e.g., activated T-cells, CD8+ T-cells, CD4+ T cells) B-cells, and/or NK cells within the live tissue sample. In some embodiments, the immune cells are T-cells. In some embodiments, the cells are cytotoxic T-cells. In some embodiments, the cells are CTLs. The term “cytotoxic T-cell” is used interchangeably with “killer T-cell” and refers to a subset of T-cells that attack and/or destroy cellular entities when activated by an antigen. Other types of T-cells include helper T-cells and regulatory T-cells. Cytotoxic T-cells are also referred to as CD8+ T-cells.

In some aspects, the assessment is an ex vivo assessment. In some embodiments, the assessment is contemporaneous (e.g., simultaneous) or nearly-contemporaneous (e.g., within 0.001 seconds; within 0.01 seconds; within 0.1 seconds; within 1 second; with 2 seconds; within 5 seconds; within 1 minute; within 5 minutes; within 30 minutes; within 45 minutes; within 1 hour, within 90 minutes; within 2 hours; within 5 hours; within 12 hours; within 1 day; within 3 days; etc.). In some embodiments, the assessment occurs over any period of time (e.g., 0.01 second, 0.05 seconds, 0.1 seconds, 0.5 seconds, 1 second, 10 seconds, 20 seconds, 1 minute, 1 hour, 1 day, 1 month, 1 year, etc.).

The methods provided herein enable efficient ex vivo contemporaneous or nearly-contemporaneous assessment of cell state within a biological sample, and enable investigation of the biological sample over time. Moreover, the methods provided herein provide for multiplexed longitudinal investigation (e.g., not a terminal or end-point investigation) of a live cell fraction within a biological sample and determining the metabolic state of the identified live cell fraction at a plurality of time points (e.g., 1, 2, 3, 4, 5, 6, 7, 8, 9, 10, 15, 20, 50, 75, 100, 150, 500, 1,000, 10,000, etc). The methods provided herein enable evaluation with single cell resolution.

In some embodiments, at least one imaging modality of the one or more imaging modalities is configured for imaging with spatial resolution equal to or less than 20 μm (such about 20 μm, 10 μm, 5 μm, 2 μm, 1 μm, 0.5 μm, 0.2 μm, 0.1 μm and the like) in a 3-dimensional array of points (voxels). In some embodiments, at least one imaging modality is configured for imaging with spatial resolution less than or equal to 2 μm. In some embodiments, at least one imaging modality is configured for imaging with submicron level spatial resolution (such as 0.1 to 0.999 μm) in a 3-dimensional array of points. In some embodiments, at least one imaging modality is configured to image at different depths of the tissue fragments. In some embodiments, at least one imaging modality is configured for non-destructive imaging of the biological sample. In some embodiments, at least one imaging modality is configured to image live biological samples (such as biological samples that have not been subjected to any fixation techniques or not been stored under any condition or for any duration of time to significantly reduce the number of viable cells). In some embodiments, at least one imaging modality is configured to image unstained biological sample (e.g., biological sample which are not labelled with any exogenous agent).

In some embodiments, one or more imaging modalities are part of an imaging system. In some embodiments, the imaging system comprises at least two imaging modalities.

In some embodiments, at least one imaging modality of the one or more imaging modalities is optical coherence microscopy (OCM) and/or dynamic optical coherence microscopy (dOCM). OCM and dOCM are enhanced interferometric techniques employing principals of optical coherence tomography (OCT) (Huang D et al. Optical coherence tomography, Science 254, 1178-1181 (1991)) to provide cross-sectional images of tissue based upon intrinsic contrasting of back-scattered light. OCT employs a raster scanned near IR beam in the lateral plane to obtain three-dimensional (3D) images of tissue structure. OCT contrast originates from back scattering between tissue layers due to difference in refractive indices. Therefore, unlike techniques which rely on fluorescent endogenous or exogenous molecules, OCT provides a label free method for contrast imaging.

The lateral resolution of traditional OCT is typically above 10 μm. However, OCM, a microscopic variant of OCT, can visualize cellular structures and achieve 1-3 μm lateral resolution by incorporating high numerical aperture objective lenses. The typical optical resolution of OCM is 1-3 μm laterally and −1 μm axially. Unlike other imaging techniques such as confocal microscopy, OCM is a non-invasive imaging approach that provides sample imaging at a high resolution without requiring fluorescent markers and high laser power, and thus avoids the possibility of causing short and long-term photodamage to the tissue.

Dynamic-OCM (dOCM) utilizes OCM technology to capture a time series of tissue dynamics (C. Apelian, F. Harms, O. Thouvenin, and A. C. Boccara, “Dynamic full field optical coherence tomography: subcellular metabolic contrast revealed in tissues by interferometric signals temporal analysis,” Biomed. Opt. Express 7(4), 1511 (2016)). Intracellular organelles (e.g., mitochondria) are highly dynamic in live cells and their metabolic activities give rise to the intracellular dynamics in live tissues. In contrast, dead tissues lack intracellular dynamics due to the absence of metabolic activities. Therefore, the dynamics in back scattered light from live and dead tissues exhibit different signatures. By analyzing the frequency components in the power spectrum of a time series of OCM images, specific frequency bands (such as 0.14 Hz-1 Hz) can be used to yield dOCM contrast for live cells. Moreover, since OCM provides high-resolution depth-resolved images, dOCM can be used to highlight live cells in 3D volumes.

In some embodiments, the methods provided herein comprise imaging a biological sample with OCM and/or dOCM to identify a live cell fraction of the biological sample. In some embodiments, identifying the “live cell fraction” or “live cell portion” or “live cell region” of the sample implies that a majority of cells in the given region of the biological sample are live, such as more than 50% of the cells are live, such as more than 55% of the cells are live, such as more than 60% of the cells are live, such as more than 70% of the cells are live, such as more than 75% of the cells are live, such as more than 80% of the cells are live, such as more than 90% of the cells are live and so on. Live cells have higher magnitudes of fluctuations at 0.14 Hz-1 Hz than dead cells, which can be detected by dOCM. Without wishing to be bound by theory, it is possible that metabolic processes occurring in live cells result in organelle movement within these living cells, which yields contrast in the dOCM image in sections of tissue having a high percentage of living cells. In contrast, these metabolic processes are absent in dead cells, and therefore dead cells lack fluctuation at 0.14 Hz-1 Hz and thus generate less contrast (e.g. a darker) in an image obtained by OCM/dOCM. Accordingly, in some embodiments the live cell fraction of the biological sample is identified as a portion of an image obtained by OCM and/or dOCM having a higher level of contrast, whereas the dead cell fraction of the image is darker (e.g. has less contrast).

In some embodiments, the methods provided herein comprise imaging a biological sample with OCM and/or dOCM to identify tissue types with different refractive indices, such as tissue type, cell density in the tissue, and tumorous/normal regions. In some embodiments, the methods provided herein comprise imaging a biological sample with OCM and/or dOCM, thereby identifying a live cell fraction in the biological sample as described above, and imaging the biological sample with multi-channel fluorescence lifetime imaging microscopy (FLIM) to classify cell state within the live cell fraction of the biological sample. In some embodiments, at least one imaging modality is configured for fluorescence imaging. In some embodiments, the imaging modality is configured for multiphoton excitation (either 2 or 3 photon excitation) of fluorescent molecules. In some embodiments, the imaging modality is configured to accomplish three-dimensional imaging using confocal fluorescence imaging or multi-photon imaging employing the use of a scanned plane of light (using 1, 2, or 3 photon excitation of fluorescent molecules). In some embodiments, the imaging modalities configured for fluorescence imaging comprises a light source (e.g., laser such as a pulsed laser employing a Titanium Sapphire gain medium and optics to generate an ultrafast pulse, or a picosecond pulsed laser emitting light in the visible spectral region of 380-700 nm, a picosecond laser emitting light in the near IR region of 700-2500 nm, or any ultrafast pulsed laser of pulse duration 30 to 500 fs), a scanner (e.g. one or more galvanometer mirror(s), or a rotating polygon mirror, or a resonant galvanometer mirror, and the like), and a detector (e.g., a charge coupled device (CCD), a CMOS based detector, an avalanche photodiode or a photomultiplier tube (PMT, or a hybrid PMT) and the like). In some embodiments, the fluorescence imaging modality comprises a light source configured to excite with an excitation wavelength in the range of preferably between 600 to 1700 nm. In some embodiments, the fluorescence imaging modality is configured for imaging with micron (e.g., 1 to 20 μm) and/or submicron (e.g., 100-999 nanometer, that is 0.1 to 0.999 μm) level spatial resolution in a 3-dimensional array of points (voxels). In some embodiments, the fluorescence imaging modality is configured to detect intrinsic emission (such as autofluorescence of molecules naturally present in biological tissue, such as intrinsic fluorophores). In some embodiments, the imaging modality is configured to detect second harmonic scattered light generated by components in the tissue fragments. In some embodiments, second harmonic scattered light is generated as the components interact with ultrafast pulsed laser light (30 to 500 fs) or pulsed laser light of picosecond pulse duration which subsequently propagate both through the sample and which are scattered back towards the excitation laser source.

Multi-photon microscopy (MPM) is an imaging technique that uses pulsed near infrared laser light to excite various endogenous fluorophores or exogenous fluorescent molecules to elucidate physical three-dimensional structure and perform spectroscopic measurements on voxels in the image field in three dimensions. MPM can be used to image endogenous fluorophores, which include molecules ranging from retinol to connective tissue (e.g., elastin, or collagen), to molecules involved in metabolic processes in the cell.

The ultrafast pulsed laser source employed in MPM enables a technique called fluorescent lifetime imaging microscopy (FLIM). FLIM is a technique that does not rely on the absolute amount of light emitted through fluorescence to make determinations about the state of a molecule, but rather, on the amount of time an electron stays in an excited state before emitting light. This means that the technique is not dependent on the number of photons present and is uniquely suited to some of the issues encountered in traditional intensity imaging of tissue samples. However, in samples with large degrees of heterogeneity due to the presence of many cell types or metabolic states associated with healthy stromal tissue, immune cells, and cancer tissue, the use of multi-channel FLIM reaches a limit due to the initial presence of many cellular metabolic states as well as molecules and complex features associated with connective tissue which also contribute to observed FLIM parameters. Primary human live tumor fragments derived from excised tumors, resections, and biopsies, and patient derived xenografts typically display high heterogeneity, and accordingly methods employing FLIM alone are not sufficient to evaluate cell state with a high degree of accuracy within such heterogenous samples.

In some embodiments the imaging modality is configured to measure the fluorescence lifetime of intrinsic fluorophore labels using one or more approaches (hereafter Fluorescent Lifetime Imaging or FLIM) (e.g., time correlated single photon counting, frequency domain methods, gated detection of photons, and the like). In some embodiments the imaging modality is configured to detect the polarization of emitted and scattered fluorescent light. In some embodiments, excitation light, which is circularly or elliptically polarized is used to perform Mueller matrix imaging. In some embodiments the generated images are deconvolved to enhance image resolution.

In some embodiments, the methods described herein involve imaging a biological sample using FLIM. In some embodiments, FLIM comprises multi-channel FLIM. In some embodiments, multi-channel FLIM provides a signal from a first intrinsic fluorophore and a signal from a second intrinsic fluorophore. In some embodiments, the “signal” provided by multi-channel FLIM comprises a set of fluorescence parameters for the first intrinsic fluorophore and the second intrinsic fluorophore. In some embodiments, the set of fluorescence parameters includes one or more of fluorescence intensity (e.g., photon counts), fluorescence lifetimes parameters (e.g., individual lifetimes of the fluorescence lifetime components, mean lifetime etc.) or fluorescence lifetime composition (e.g., relative amplitudes of the fluorescence lifetime components etc.) of one or more fluorophores (such as intrinsic fluorophores). In some embodiments a set of fluorescence parameters comprises, photon counts from the intrinsic fluorophore, a weighted mean (τₘ) of the lifetimes of a plurality of fluorescence lifetime components of the intrinsic fluorophore and the amplitude (aₙ) of at least one fluorescence lifetime component relative to the plurality of fluorescence lifetime components. In some embodiments, the set of fluorescence parameters further comprises individual lifetimes of at least two fluorescence lifetime components of the plurality of fluorescence lifetime components.

In some embodiments, an intrinsic fluorophore comprises n, (wherein n>1) fluorescence lifetime components distinguished by fluorescence lifetimes. In some embodiments, the fluorescence lifetime (or lifetime) of the 1^{st} fluorescence lifetime component is τ₁, the lifetime of the 2^{nd} fluorescence lifetime component is τ₂, the lifetime of the n^{th} fluorescence lifetime component is r.. In some embodiments, the amplitude of the 1^{st} fluorescence lifetime component is ai, the amplitude of the 2^{nd} fluorescence lifetime component is a₂, the amplitude of the n^{th} fluorescence lifetime component is an. In some embodiments, τₘ is the weighted mean of τ₁ to τₙ for an intrinsic fluorophore with n fluorescence lifetime components.

Any suitable first and second fluorophore may be used. Exemplary intrinsic fluorophores include molecules ranging from retinol to connective tissue (e.g., elastin, or collagen), to molecules involved in the energy creation processes in the cell. For example, the metabolic cofactor molecules nicotinamide adenine dinucleotide (NAD(P)H in its reduced form, the P denoting phosphorylation) and flavin adenine dinucleotide (FAD) are ubiquitous in all cells and dominate the naturally occurring fluorescent signal on a cellular level, and therefore may be used as intrinsic fluorophores in the methods described herein. FAD is a protein bound metabolic electron carrier with a reduced (FADH₂) and oxidized (FAD) form. NADH, a pyridine nucleotide serving as a major metabolic electron carrier, exists in a reduced (NADH), oxidized (NAD′), and phosphorylated (NADPH or NADP′) form throughout the cell. NADH and oxidized and phosphorylated forms thereof may be used as an intrinsic fluorophore.

In some embodiments, the first intrinsic fluorophore is NAD(P)H and the second intrinsic fluorophore is FAD. In some embodiments, the signal from the first intrinsic fluorophore and the signal from the second intrinsic fluorophore are identified by phasor segmentation analysis (Digman et al., The Phasor Approach to Fluorescence Lifetime Imaging Analysis, Biophysical Journal, Volume 94, Issue 2, 15 Jan. 2008, Pages L14-L16).

In some embodiments, the methods described herein comprise calculating a lifetime metabolic ratio (LMR) of the live cell fraction. For example, in some embodiments assessing cell state within the live cell fraction comprises calculating the LMR for the live cell fraction. As another example, in some embodiments determining a baseline metabolic signature for the live cell fraction comprises calculating the LMR for the live cell fraction. In some embodiments, the baseline metabolic signature (e.g. LMR) serves as a basis from which to measure change in metabolic activity within the live cell fraction, such as following addition of a potential therapeutic agent.

In some embodiments, the LMR is calculated by dividing a signal from the first intrinsic fluorophore corresponding to the amount of enzyme-bound NAD by a signal from the second intrinsic fluorophore corresponding to the amount of enzyme-bound FAD. This calculation is represented mathematically as:

$$\frac{{{α}_{2} [ \% ]}_{NAD ( P ) H}}{{{α}_{1} [ \% ]}_{FAD}} .$$

In some embodiments, the LMR is indicative of cellular activity in the sample. In some embodiments, the LMR is indicative of relative glycolytic vs. oxidative phosphorylation activity of cells in the sample. For example, in some embodiments increased oxidative phosphorylation in cells present within the biological sample leads to an increase in the amount of enzyme-bound NAD in the sample, which would increase the LMR calculated. In contrast, decreased oxidative phosphorylation or a shift from oxidative phosphorylation to glycolysis would lead to a decrease in the LMR calculated. In some embodiments, increase in the LMR is indicative of the generation of reactive oxygen species (ROS) associated with apoptotic mechanisms, decrease of mitochondrial cell health, and mechanisms leading to cell death. In some cases, the decrease in the LMR is associated with loss of membrane integrity and necroptotic mechanisms leading to cell death.

In some embodiments, the signal from the first intrinsic fluorophore (e.g., NAD(P)H) and the signal from the second intrinsic fluorophore are obtained via two different channels at different excitation wavelengths. Suitable excitation wavelengths range from 360 to 900 nm. In some embodiments, the excitation wavelength for NAD(P)H is about 740 nm and the excitation wavelength for FAD is about 880 nm.

In some embodiments, provided herein are methods of assessing (e.g., evaluating) (e.g., characterizing) a biological sample. In some embodiments, provided herein is a method of assessing (e.g., evaluating) (e.g., characterizing) a biological sample comprising imaging a biological sample with OCM and/or dOCM, thereby identifying a live cell fraction of the biological sample, and imaging the biological sample with fluorescence lifetime imaging microscopy (FLIM) to determine a baseline metabolic signature for the live cell fraction of the biological sample. In some embodiments, imaging the biological sample with FLIM comprises imaging the live cell fraction identified with OCM and/or dOCM. In some embodiments, imaging the biological sample with FLIM comprises imaging the entire biological sample.

In some embodiments, additional imaging modalities can be utilized along with OCM/dOCM and FLIM within methods for assessing cell state within a biological sample. For example, in some embodiments, methods are provided for assessing cell state within a biological sample through use of OCM/dOCM and FLIM, and optionally, one or more additional imaging modalities. Non-limiting examples of additional imaging modalities include for example mass spectrometry (MS), nuclear magnetic resonance spectroscopy, magnetic resonance imaging, light sheet microscopy, lattice light sheet microscopy, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography), SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonies generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

In some aspects, such assessment (e.g., ex vivo contemporaneous or nearly-contemporaneous assessment) of a biological sample can provide relevant information about the effect of any type or kind of intervention on the biological sample. For example, in some aspects, provided herein are methods of assessing an intervention or plurality of interventions. The methods comprise 1) imaging a biological sample with OCM/dOCM, thereby identifying a live cell fraction of a biological sample, and imaging the biological sample with FLIM to determine a baseline metabolic signature, 2) contacting the biological sample with one or more (e.g., same or different) interventions, 3) re-imaging the biological sample with: both OCM/dOCM and FLIM, OCM/dOCM alone, or FLIM alone, and 4) evaluating the biological sample (e.g., live cell fraction) in the sample for purposes of evaluating the effect of the one or more interventions (e.g., detecting changes in the cell state prior to and after exposure to the one or more interventions).

In some aspects, provided herein are methods of evaluating one or interventions (e.g., identical or non-identical interventions). In some embodiments, the methods comprise imaging a biological sample with OCM/dOCM, thereby identifying a live cell fraction of a biological sample, and imaging the biological sample with FLIM to determine a baseline metabolic signature, as described above. Such methods further comprise exposing the biological sample to an intervention and measuring a metabolic shift in the live cell fraction from a baseline metabolic signature for the live cell fraction. In some embodiments, the baseline metabolic signature for the live cell fraction is determined by calculating the LMR for the live cell fraction, as described above. In some embodiments, the methods further comprise classifying cell state within the live cell fraction based upon the metabolic shift measured. In some embodiments, dOCM methods identify a live cell fraction and are used in conjunction the LMR signatures to define LMR signatures of individual healthy (live) cells in the biological sample. In some embodiments, metabolic shifts in the LMR of individual cells from healthy states are used to determine the individual cell state changes over a time course. In some embodiments, dOCM methods are used in conjunction with LMR signatures to define dead cells in time course observation.

In some aspects, provided herein are methods of assessing a potential therapeutic agent or a combination of different potential therapeutic agents. The methods comprise 1) imaging a biological sample with OCM/dOCM, thereby identifying a live cell fraction of a biological sample, and imaging the biological sample with FLIM to determine a baseline metabolic signature, 2) contacting the biological sample comprising the identified cell fraction with one or more (e.g., same or different) therapeutic agents, 3) re-imaging the biological sample with: both OCM/dOCM and FLIM, OCM/dOCM alone, or FLIM alone, and 4) evaluating the biological sample (e.g., live cell fraction) in the sample for purposes of evaluating the effect of the one or more therapeutic agents (e.g., detecting changes in the cell state prior to and after exposure to the potential therapeutic agent or combination of therapeutic agents).

In some aspects, provided herein are methods of evaluating one or more potential therapeutic agents (e.g., identical or non-identical potential therapeutic agents). In some embodiments, the methods comprise imaging a biological sample with OCM/dOCM, thereby identifying a live cell fraction of a biological sample, and imaging the biological sample with FLIM to determine a baseline metabolic signature, as described above. Methods of evaluating a potential therapeutic agent further comprise exposing the biological sample to a potential therapeutic agent and measuring a metabolic shift in the live cell fraction from a baseline metabolic signature for the live cell fraction. In some embodiments, the baseline metabolic signature for the live cell fraction is determined by calculating the LMR for the live cell fraction, as described above. In some embodiments, the methods further comprise classifying cell state within the live cell fraction based upon the metabolic shift measured. In some embodiments, dOCM methods identify a live cell fraction and are used in conjunction the LMR signatures to define LMR signatures of individual healthy (live) cells in the biological sample. In some embodiments, metabolic shifts in the LMR of individual cells from healthy states are used to determine the individual cell state changes over a time course. In some embodiments, dOCM methods are used in conjunction with LMR signatures to define dead cells in time course observation.

In some aspects, the assessment (e.g., assessment of one or more interventions) (e.g., assessment of one or more potential therapeutic agents) is contemporaneous (e.g., simultaneous) or nearly-contemporaneous (e.g., within 0.001 seconds; within 0.01 seconds; within 0.1 seconds; within 1 second; with 2 seconds; within 5 seconds; within 1 minute; within 5 minutes; within 30 minutes). In some aspects, the assessment is staggered over several time points. In some aspects, the assessment is staggered over longer time points (e.g., 1 hour, 2 hours, 6 hours, 12 hours, 1 day, 2 days, 1 week, 1 month, etc.).

In some embodiments, the metabolic shift is measured by FLIM. In some embodiments, the metabolic shift is measured by multi-channel FLIM. For example, in some embodiments the metabolic shift is measured by multi-channel FLIM using a first intrinsic fluorophore and a second intrinsic fluorophore. In some embodiments metabolic shift is measured by multi-channel FLIM using a first intrinsic fluorophore, a second intrinsic fluorophore, and a third intrinsic fluorophore. In some embodiments, the metabolic shift is measured by multi-channel FLIM using a first intrinsic fluorophore, a second intrinsic fluorophore, and a third extrinsic fluorophore. In some embodiments the first and second intrinsic fluorophores are the same fluorophores used in the determination of the baseline metabolic signature (e.g., baseline LMR value) for the biological sample. For example, in some embodiments the methods first comprise obtaining a baseline metabolic signature for the live cell fraction, as described above. In some embodiments, the baseline metabolic signature is obtained by multi-channel FLIM using a first intrinsic fluorophore (e.g. NAD(P)H) and a second intrinsic fluorophore (e.g., FAD), obtaining a signal from each fluorophore (e.g., by phasor segmentation analysis), and calculating the LMR in the live cell fraction as described above. The LMR calculated prior to addition of the potential therapeutic agent is also referred to as the “baseline LMR” herein. In some embodiments, the metabolic shift is then measured in the sample after contacting the sample with the potential therapeutic agent, wherein the metabolic shift is measured by calculating the LMR value for the sample using the signals obtained from the first intrinsic fluorophore (e.g., NAD(P)H) and the second intrinsic fluorophore (e.g., FAD). In some embodiments, the signals from the first intrinsic fluorophore and the second intrinsic fluorophore used to determine the metabolic shift in the sample are obtained by phasor segmentation analysis. In some embodiments, a first intrinsic fluorophore (e.g. NAD(P)H) and a second intrinsic fluorophore (e.g., FAD), and a third intrinsic fluorophore (e.g. lipofuscin) can be used to determine metabolic state. In some embodiments, a first intrinsic fluorophore (e.g. NAD(P)H) and a second intrinsic fluorophore (e.g. FAD), and a third added extrinsic fluorophore (e.g. propidium iodide, or a sensor probe for caspase 3 or 7 activity) can be used to determine metabolic state in the sample.

In some embodiments, the methods comprise classifying cell state within the live cell fraction based upon the metabolic shift measured. In some embodiments, a metabolic shift above a threshold value is indicative of cell death. For example, in some embodiments a metabolic shift above a threshold value indicates that cell death is occurring within the region previously identified as the live cell fraction of the sample. In some embodiments, the cell death is occurring due to the potential therapeutic agent used to treat the sample. In some embodiments, cell death is via apoptosis. In some embodiments, cell death is via necroptosis. In some embodiments, cell death is via a combination of apoptosis and necroptosis.

In some embodiments, the methods described herein can be used to differentiate between apoptotic cell death and necroptotic cell death in a sample. For example, in some embodiments the methods of evaluating a potential therapeutic agent described herein differentiate between whether the agent induces cell death via apoptosis or via necroptosis in the sample. In some embodiments, a metabolic shift within a first range of reference values is indicative of apoptosis. In some embodiments, a metabolic shift within a second range of reference values is indicative of necroptosis.

In some embodiments, any of the methods of assessing an intervention, a combination of interventions, a potential therapeutic agent or a combination of different therapeutic agents described herein further include utilization of one or more additional imaging modalities.

Examples of potential interventions include, but are not limited to, potential therapeutic agents, potential non-therapeutic agents (e.g., deleterious agents), apoptosis markers (e.g., a caspase selected from the group consisting of caspase 3, 6, 7, 8, and 9, and caspase 3/7) (e.g., a granzyme selected from the group consisting of granzyme A, B, C, D, E, F, G, H, K, and M), an apoptosis inducing agent (e.g., staurosporine), a necroptosis inducing agent (e.g., sorafenib), cell fractions from unhealthy tissue, cell fractions from healthy tissue, viruses, bacteria, pathogens, food and food products or components, endogenous molecules (e.g., gene expression products or hormones that are artificially induced), metabolites, radiation, heat, cold, oxygen or other gases, and the like.

The methods described herein are broadly applicable to assessing any potential therapeutic agent. The term “potential therapeutic agent” or “agent” refers to any compound, material, substance, or condition that provides a potential therapeutic benefit, either alone, or in combination with other therapeutic approaches. Examples of potential therapeutic agents include, but are not limited to, a small molecule, a polypeptide or peptide fragment, an siRNA, a cellular therapy (e.g., tumor-infiltrating lymphocyte therapy, CAR T-cell therapy, T-cell receptor-based therapy, natural killer cell therapy, etc.), an oncolytic virus, a metabolite treatment (e.g., an adenosine treatment), or an antibody or fragment thereof. Agents need not be drugs. Agents include, but are not limited to, food and food products or components, endogenous molecules (e.g., gene expression products or hormones that are artificially induced), metabolites, radiation, heat, cold, oxygen or other gases, and the like. In some embodiments, the “agent” is a biological agent such as an oncolytic virus (such as talimogene laherparepvec) or a cellular therapy (such as chimeric antigen receptor (CAR)-T cell). In some embodiments, the agent is a cell or a plurality of cells, such as one or more leukocyte (e.g. white blood cell). For example, in some embodiments the potential therapeutic agent is a leukocyte, such as a B-cell, NK-cell, T-cell, macrophage, neutrophil, eosinophil, basophil, etc. In some embodiments, the potential therapeutic agent is an activated cell (e.g. an activated T-cell). In some embodiments, the potential therapeutic agent comprises multiple cell types.

In some embodiments, the potential therapeutic agent is a cell, wherein the cell is intended for use as an anti-cancer therapy. Accordingly, in some embodiments the methods of evaluating the potential therapeutic agent (e.g. the cell) identify whether the cell is an effective anti-cancer therapy based upon the metabolic shift measured in the biological sample following contacting the sample with the cell. In some embodiments, the sample is a tumor sample, and the agent is identified as an effective anti-cancer therapy if the cell induces a metabolic shift indicative of cell death. For example, in some embodiments the agent is identified as an effective anti-cancer therapy when the cell induces a shift in the LMR indicative of apoptosis and/or necroptosis in the sample.

In some embodiments, the potential therapeutic agent is a drug. As used herein, the term “drug” is used in the broadest sense and refers to any agent or medicine with potential use as a therapeutic. For example, a drug can be an antibody or a fragment thereof, an aptamer, a protein, a nucleic acid (e.g. DNA, RNA, siRNA, shRNA), a small molecule, a compound, and the like. In some embodiments, the drug is an anti-cancer drug. In some embodiments, the anti-cancer drug is a targeted anti-cancer agent, such as a targeted antibody (such as anti-her2 antibody), an antibody fragment, bispecific antibody (such as bispecific T cell engager or BiTe), antibody-drug conjugates (such as trastuzumab emtansine), antibody-dependent cell cytotoxicity (ADCC) related to monoclonal antibody (mAb)-mediated therapy, or a targeted small molecule (e.g., protein inhibitor, such as kinase inhibitor).

In some embodiments, the anti-cancer drug is a cytostatic or cytotoxic agent, non-limiting examples of which include adriamycin, gemcitabine, palbociclib, docetaxel, fulvestrant, alpelisib, trametinib, cisplatin, carboplatin, oxaliplatin, exemestane, everolimus, vinorelbine, olaparib, capecitabine, cyclophosphamide, methotrexate, fluorouracil, mitomycin C, temozolomide, cetuximab, trastuzumab, leucovorin, topotecan, irinotecan and any combination thereof. In some embodiments, the anti-cancer drug is an immunotherapeutic agent or drug, non-limiting examples of which include an immune checkpoint inhibitor or an immunostimulatory agent. In some non-limiting embodiments, the immunotherapeutic drug includes nivolumab, ipilimumab, pembrolizumab, atezolizumab, and any combination thereof. In some embodiments, the drug is an interfering RNA, such as small interfering RNA (siRNA) or short hairpin RNA (shRNA). In some embodiments, the drug is a potential anti-cancer agent, and the methods of evaluating the potential therapeutic agent (e.g. the drug) identify whether the drug is an effective anti-cancer therapy based upon the metabolic shift measured in the biological sample following contacting the sample with the cell. In some embodiments, the sample is a tumor sample, and the agent is identified as an effective anti-cancer therapy if the drug induces a metabolic shift indicative of cell death. For example, in some embodiments the agent is identified as an effective anti-cancer therapy when the drug induces a shift in the LMR indicative of apoptosis and/or necroptosis in the sample.

In other embodiments, the drug is a potential therapeutic agent, and the methods described herein can be used to assess whether the agent induces unwanted toxicity in normal tissue. In such embodiments, toxicity of the agent can be measured by assessing the metabolic shift in the biological sample after contacting the sample with the agent. In such embodiments, a metabolic shift (e.g. a shift in LMR) indicative of cell death, such as via apoptosis and/or necroptosis, would indicate that the agent is toxic to the sample.

In some embodiments, software is provided for assessing cell state within the live tissue sample. In some embodiments, the software is configured to interpret cell state within a biological sample. In some embodiments, the software is configured to compare cell state within a biological sample to established norm controls for various cellular responses (e.g., cellular state associated with a healthy response, abnormal response, etc.).

For example, in some aspects, methods are provided for assessing a biological sample, comprising providing a biological sample, and a processor comprising software that, when executed, causes the processor to manually or automatically: image the biological sample with OCM and/or dOCM to identify a live cell fraction in the biological sample; and image the biological sample (e.g., identified live cell fraction) with FLIM to classify the cell state within the identified live cell fraction; and provide a report to a user of the identified and classified live cell fraction; executing the processor to image the biological sample with OCM and/or dOCM to identify a live cell fraction in the biological sample; and image the identified live cell fraction with FLIM to classify the cell state within the identified live cell fraction, and provide a report to a user of the identified and classified live cell fraction.

In some aspects, methods are provided for assessing one or more potential therapeutic agents, comprising providing a biological sample, and a processor comprising software that, when executed, causes the processor to manually or automatically: image the biological sample with OCM and/or dOCM to identify a live cell fraction in the biological sample; image the biological sample (e.g., identified live cell fraction) with FLIM to classify the cell state within the identified live cell fraction; contact an identified and classified live cell fraction with one or more potential therapeutic agents after an identification and classification of a live cell fraction with OCM and/or dOCM and FLIM; image the biological sample with OCM/dOCM and/or FLIM to re-classify the cell state within the identified and classified live cell fraction; measure classification differences pre- and post-contacting of identified and classified live cell fraction with the one or more potential therapeutic agents; provide a report to a user of one or more of: an identified and classified live cell fraction; a re-classified identified and classified live cell fraction; and classification differences pre- and post-contacting of identified and classified live cell fraction with the one or more potential therapeutic agents; executing the software to image the biological sample with OCM and/or dOCM to identify a live cell fraction in the biological sample; image the identified live cell fraction with FLIM to classify the cell state within the identified live cell fraction; contact an identified and classified live cell fraction with one or more potential therapeutic agents after an identification and classification of a live cell fraction with OCM and/or dOCM and FLIM; image the biological sample (e.g., identified live cell fraction) with OCM/dOCM and/or FLIM to re-classify the cell state within the identified and classified live cell fraction; measure classification differences pre- and post-contacting of identified and classified live cell fraction with the one or more potential therapeutic agents; provide a report to a user of one or more of an identified and classified live cell fraction; a re-classified identified and classified live cell fraction; and classification differences pre- and post-contacting of identified and classified live cell fraction with the one or more potential therapeutic agents.

In some aspects, the methods described herein are performed at least in part using machine learning. For example, in some embodiments a machine learning classifier is used to determine a live cell reaction of a biological sample. As another example, in some embodiments a machine learning classifier is used to assess cell state within the live cell region of a biological sample. In some embodiments, a machine learning classifier is used to determine whether cell death is occurring in the live cell region of the biological sample (e.g. as a result of contacting the sample with a potential therapeutic agent). Non-limiting examples of machine learning classifiers include, a gradient boosting classifier, a random forest classifier, or a deep learning classifier, including a convolutional neural network (CNN).

In some embodiments, the machine learning classifier is trained on a set of reference biological samples labelled with a first exogenous label (or exogenous agent used interchangeably) that stains apoptotic cells and a second exogenous agent that stains dead cells. Non-limiting examples of first exogenous label that stains apoptotic cells comprises labels (such as exogenous agent or fluorophores) that stain caspase3/7. Non-limiting examples of second exogenous label that stains dead cells comprises agents like propidium iodide that can penetrate the membrane of dead cells.

In some embodiments, the biological sample utilized in the above described methods for assessing cell state includes live tissue fragments. Suitable methods for producing a biological sample comprising live tissue fragments (e.g., live tumor fragments) are described in U.S. patent application Ser. No. 17/566,154, the entire contents of which are incorporated herein by reference for all purposes. Generally speaking, live tumor fragments may be obtained by obtaining a tumor containing tissue sample from a subject, preserving/preparing the tissue as necessary for slicing, slicing the tissue into appropriate sizes under appropriate conditions to prevent a reduction in cell viability in the tissue, and maintaining the tissue under suitable conditions to maintain cell viability.

In some embodiments, the tissue, after being obtained from the subject, is first cut into tissue fragments. In some embodiments, the tissue fragments are placed in a suitable medium for extended preservation of cell viability, such as for transportation to a laboratory, where further processing of the tissue fragments takes place (such as sorting, imaging, culture etc.). In some embodiments, the tissue fragments are preserved under hypothermic preservation conditions. The term “hypothermic preservation” or “hypothermal preservation” mean preservation at a temperature below the physiological temperature (which is about 37° C.) but above the temperature of freezing, wherein biological processes are slowed down, thus allowing prolonged storage of a biological material. In some embodiments, hypothermic preservation is performed at temperatures between about 0° C. and about 10° C. A “hypothermally preserved tissue” or a “hypothermally preserved tissue fragment” refers to a tissue or a tissue fragment respectively, that has been preserved under hypothermic conditions. The terms “hypothermic preservation” and “cold preservation” have been used interchangeably. Similarly, the terms “hypothermic transport” and “cold transport” have been used interchangeably.

In some embodiments, the live tissue fragments and/or live tumor fragments are preserved under cryopreservation conditions (such as at sub-zero temperature). The term “cryopreservation” means preservation of a biological material (such as tissue or tissue fragment) at a temperature below the freezing temperature (such as at sub-zero temperature). A “cryopreserved tissue” or a “cryopreserved tissue fragment” refers to a tissue or a tissue fragment respectively, that has been preserved at temperature below the freezing temperature (such as at sub-zero Celsius temperature). A sub-zero Celsius temperature (or sub-zero temperature) is any temperature below 0° C., such as less than about −10° C., less than about −20° C., less than about −50° C., less than about −100° C., less than about −120° C., less than about −150° C. and so on. In some embodiments, sub-zero temperature is a temperature of liquid nitrogen, such as the boiling temperature of liquid nitrogen at atmospheric pressure. In some embodiments, sub-zero temperature is a temperature between about 0° C. and about −200° C. In some embodiments, sub-zero temperature is a temperature of about −196° C. In some embodiments, the live tissue fragments and/or live tumor fragments are thawed for subsequent processing on reaching the destination site, such as a laboratory, where subsequent processing of the tissue fragments take place. In some embodiments, the tissue fragments are preserved under conditions, wherein after thawing, the viability of cells in the tissue fragments is not significantly reduced. In some embodiments, the tissue fragments are preserved under conditions, wherein after thawing, the viability of cells in the tissue fragments is significantly reduced. In some embodiments, preservation of the tissue fragments under cryopreservation or hypothermic preservation conditions allows the tissue fragments to be stored for extended periods of time without significant reduction in cell viability or alterations in its metabolic profile. This allows great flexibility in the workflow and logistics. For example, it obviates any restriction of distance between the source site of tissue (such as a hospital) and the destination site (such as a laboratory) or of time elapsed between excision of the tissue and initiation of culture.

In some embodiments, the tissue is placed in a suitable medium for preservation before it is cut into tissue fragments. In some embodiments, the tissue is maintained under hypothermic preservation conditions in a suitable hypothermic preservation medium or under cryopreservation conditions in a suitable cryopreservation medium. The term “hypothermic preservation medium” means a preservation composition that would allow the biological material to withstand a temperature below the physiological temperature, such as a temperature below 10° C. to sustain its viability at such temperature. The terms “cryopreservation medium”, or “freezing medium”, refer to a medium in which a biological material is immersed before cryopreservation or freezing, or to medium which can be used to treat the biological material prior to freezing. A cryopreservation medium contains one or more cryoprotectants. In certain embodiments, a cryopreservation medium may be a freezing solution, a vitrification solution, and/or a mixture of such solutions. In certain embodiments, the cryopreservation medium refers to a medium for storing or freezing a biological material at a sub-zero Celsius temperature to sustain the viability of the tissue or the tissue fragments at that temperature. In some embodiments, the hypothermally preserved or the cryopreserved tissue is transported to a destination site, such as the laboratory for further processing. In some embodiments, the tissue is cut into tissue fragments after transportation. In some embodiments, a cryopreserved tissue is thawed before being cut into tissue fragments.

For any of the embodiments described herein, cutting the tissue can be performed manually, or it can be semi-automated or automated. Various suitable cutting devices may be employed for cutting the tissue. In some embodiments, the cutting device is configured to cut the tissue precisely and with minimal mechanical damage to the tissue or the tissue fragments. In non-limiting examples, cutting devices comprise a knife, a blade, a wire, a scalpel, a laser, and the like. In some embodiments, the cutting device comprises a plurality of blades. In some embodiments, the cutting device comprises a coated wire, such as a diamond particle coated steel wire (such as a diamond wire). In some embodiments, the cutting device comprises uniformly spaced wires (such as diamond wires or naked steel wired). In some embodiments, the cutting device comprises a cutting component. In some embodiments, the cutting component comprises at least one cutting member such as a knife, a blade, a wire, a scalpel, a laser, and the like. In some embodiments, the cutting device comprises three cutting components to cut the tissue in three dimensions, wherein each cutting component cuts the tissue in one dimension. In some embodiments, the tissue sample can be rotated 90 degrees relative to a single cutting component to make two of the three dimensional cuts. In some embodiments, the single cutting component can be rotated to make two of the three dimensional cuts. The cutting device is configured to accurately and precisely cut a tissue into tissue fragments of a defined size. In some embodiments, the cutting device is configured to cut the tissue into tissue fragments based on a size input received from the user (user-defined). In some embodiments, the user-defined size input is based on physical properties of the tissue such as mechanical stiffness, frangibility and the like. In some embodiments, the cutting device is configured to cut the tissue into tissue fragments based on a pre-defined size input. In some embodiments, the cutting device is configured to cut the tissue into tissue fragments automatically and repeatedly until the entire tissue is cut into tissue fragments. In some embodiments, the cutting device is configured to cut the tissue into tissue fragments that are equal in size. As used herein, equal means substantially equal wherein the sizes of the tissue fragments are within ±20% of one another, in at least one dimension. In some embodiments, depending on the firmness of the tissue, the cutting device or components thereof are vibrated or rotated at user-defined or pre-defined frequency. The fragmentation settings of the cutting device such as thickness of tissue fragment, frequency, amplitude, speed etc. are user-defined or pre-defined.

In some embodiments, the tissue is cut under conditions of high oxygen concentration, that is an oxygen concentration greater than ambient oxygen concentration (such as greater than 21% or greater than 30% or greater than 50% or greater than 70%, or greater than 90% and the like). In some embodiments, the tissue is cut into tissue fragments in an oxygenated cutting medium.

In some embodiments, the tissue is prepared before cutting. For example, in some embodiments, the tissue is encapsulated in a gel matrix. A gel matrix can comprise a synthetic, a semi-synthetic or a natural component. In some embodiments, a gel matrix comprises at least one synthetic polymer or co-polymer, non-limiting examples of which includes poly(ethylene glycol) (PEG), poly(hydroxyethyl methacrylate) (PHEMA), poly(vinyl alcohol) (PVA), poly(acrylic acid) (PAA), poly(lactic acid), poly(caprolactone), poly(methycrylic acid) (PMMA), poly(lactic-co-glycolic acid) (PLGA), polyhydroxybutyric acid-valeric acid, poly(ethylene glycol)-diacrylate, poly(ethylene glycol)-vinyl sulfone and the like. In some embodiments, the polymers or co-polymers are further functionalized. In some embodiments, the tissue is contacted with a gel precursor. A gel precursor is a component that forms the gel matrix under suitable conditions of gelation. The gel precursor can be in any physical form such as in liquid or in solid form. In some embodiments, the gel matrix is formed by a covalent cross-linking of the gel precursors, while in some other embodiments the gel matrix is formed by a physical aggregation of the gel precursors. In some embodiments, depending upon the tissue type the percentages of the gel precursors and/or gelation conditions can be varied to obtain gel matrices of varying mechanical stiffness. In some embodiments, a gel matrix is formed when the gel precursor is irradiated with a light source. In some embodiments a gel matrix is formed when the gel precursor is subjected to a temperature change. While a skilled artisan can envisage multiple types of suitable gel matrices and gelation conditions, preferably the process of gelation to form the gel matrix should be fast and under conditions that cause minimal damage to the tissue or tissue fragments and that are inert to biological molecules. Further, the process of gelation and/or the gel matrix should not significantly alter the biological behavior of the cells in the tissue fragment. In some embodiments, gelation to form the gel matrix happens in less than 5 min (such as 4 min, 3 min, 2 min, 1 min or 30 second).

In some embodiments, the gel precursor is a PEG polymer such as a linear or a branched PEG polymer. A particularly suitable functionalized polymer can be, for example, a multi-arm, branched PEG polymer, such as a four-arm or an eight-arm PEG with terminal hydroxyl (—OH) groups that is functionalized with norbornene. In some embodiments, gelation to form the gel matrix happens in the presence of a suitable cross-linker such as a di-thiolated molecule (e.g., bi-functional PEG-dithiol). In some embodiments gel formation happens when the norbornene-functionalized multi-arm PEG polymer and bi-functional PEG-dithiol are irradiated with a light source.

In some embodiments, the tissue is contained within a sacrificial casing. While the gel matrix, the sacrificial casing, or both help to hold and stabilize the tissue during cutting, it is preferable not to have any trace of either during culture of the tissue fragments since residual gel matrix or residual sacrificial casing can interfere with nutrient availability, drug response and/or downstream analysis of the tissue fragments. In some embodiments, residual gel matrix, residual sacrificial casing or both are removed before the tissue fragments are contacted with the therapeutic agent(s). In some embodiments, the step of cutting comprises driving the sacrificial casing containing the tissue towards the cutting component of the cutting device or driving the cutting component of the cutting device towards the sacrificial casing containing the tissue, wherein the cutting device cuts the tissue into tissue fragments by cutting through the sacrificial casing. In some embodiments, the sacrificial casing is formed of a material that can be cut with a cutting mechanism. Non-limiting examples of materials of the sacrificial casing include polypropylene, wax, silicone (such as Polydimethylsiloxane (PDMS)) and various thermoplastic elastomers. The material should preferably be biocompatible and non-toxic to avoid damaging or altering the tissue properties. In some embodiments, the sacrificial casing comprises a hollow cavity to house the tissue within. In some embodiments, the sacrificial casing comprises a groove to hold the tissue.

In some embodiments, the living tissue sample is a whole biopsy tissue sample. In some embodiments, the living tissue sample is a bisected biopsy tissue sample (either longitudinal cuts that can range from 100-700 microns thick or bisected cuts that can range from 100 microns to 1 mm thick or combinations therein). In some embodiments, the living tissue sample is a living tissue fragment.

In some embodiments, the living tissue sample (e.g., whole biopsy; bisected biopsy tissue sample; living tissue fragment(s)) is maintained in suitable culture conditions within a culture platform. A culture platform is any suitable culture device or system for culturing tissue fragments. Non-limiting examples of a culture platform include a well-plate or a fluidic device. In some embodiments, the culture platform comprises an oxygen-permeable material.

Various types of oxygen-permeable materials may be employed. In some embodiments, the oxygen-permeable material comprises a fluoropolymer, non-limiting examples of which include FEP (fluorinated ethylene-propylene), TFE (tetrafluoroethylene), PFA (perfluoroalkoxy), PVF (polyvinylfluoride), PVDF (polyvinylidene fluoride), PTFE (polytetrafluoroethylene), PCTFE (polychlorotrifluoroethylene), ETFE (polyethylenetetrafluoroethylene), ECTFE (polyethylenechlororifluoroethylene), FFPM/FFKM (perfluoroelastomer), FPM/FKM (chlorotrifluoroethylenevinylidene fluoride), PFPE (perfluoropolyether), MFA (tetrafuoroethylene and perfuoromethyl vinyl-ether copolymer), CTFE/VDF (chlorotrifuoroethylene-vinylidene fluoride copolymer), and TFE/HFP (tetrafuoroethylene-hexafuoropropylene copolymer), or mixtures thereof. In some embodiments, the oxygen-permeable material comprises cyclic olefin polymer (COP) and cyclic olefin copolymers (COC). In some embodiments, the oxygen-permeable material comprises a silicone material (e.g., polydimethylsiloxane (PDMS)). In some embodiments, the culture platform is formed of extremely thin sections of one or more oxygen-permeable material. In some embodiments, regions of culture platform include chambers of the culture platform. Chambers of the culture platform can be wells of a well-plate or channels of a fluidic device. In some embodiments, the culture platform is configured for perfusion culture. In some embodiments, the culture platform is configured for non-perfused, static culture. In some embodiments, the culture platform is formed of a material that is optically transparent, thereby allowing optical investigation of the tissue fragments while the tissue fragments are within the chambers of the culture platform.

### EXAMPLES

The following examples are intended only to illustrate methods and embodiments in accordance with the invention, and as such should not be construed as imposing limitations upon the claims.

### Example 1

Live tumor fragments (LTFs) from flank expressed murine syngeneic CT26 tumors were used to assess cell state using a combination of OCM/dOCM and FLIM as described herein.

CT26 tumors were excised, bisected, and cut into 300×300×300 μm LTFs. These fragments were sorted into 18-well plates for analysis by microscopy. Fragments were incubated at 37° C. in 5% CO₂ in non-phenol red containing RPMI for up to four hours before being placed in the microscope. During imaging the sample was maintained in a stage-top incubator at 37° C., 5% CO₂, and 95% humidity. Images were taken using a custom four channel multi-photon microscope built on a modified Zeiss Axiovert platform using adapted Bruker components, the PrairieView software package, and electronic components to drive and control the scanning microscope. FLIM data was acquired using an 8 channel Swabian instruments time tagger.

FLIM images were acquired at 50 micron and 75 micron depths into the tissue. Live tumor fragment structure and metabolic status were assessed based on the intrinsically fluorescent metabolic co-factors nicotinamide dinucleotides (NAD(P)H), excited at 740 nm, and flavin adenine dinucleotide (FAD), excited at 880 nm. The resultant data were displayed as a phasor plot and the ratio of the alpha 2 decay component of NAD(P)H and the alpha 1 decay component of FAD (e.g. the LMR) were determined and plotted on a pixel-by-pixel basis in false-colored images. LMR histogram data from the fragments was normalized by taking the mode of the resultant histogram data at baseline from healthy tissue and setting this value to 0 across the time course. Data derived from the NAD(P)H, and FAD signals were used to segment the image into individual Voronoi cells seeded from nuclear segmentation. The normalized LMR value was determined for each Voronoi cell.

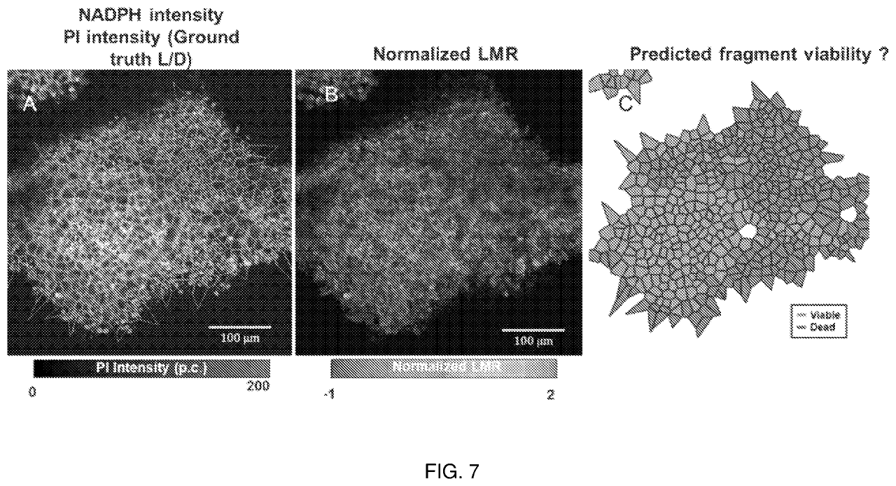

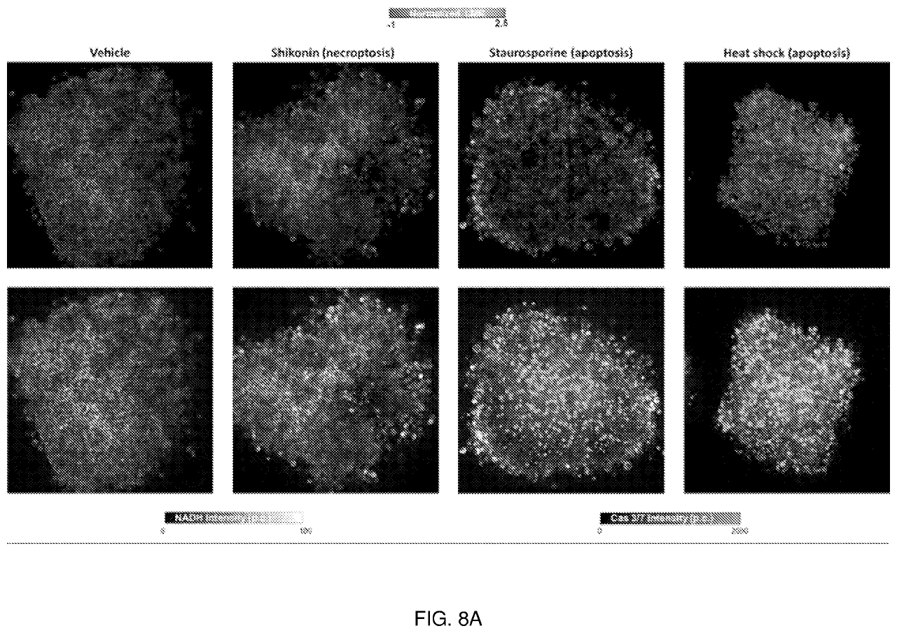

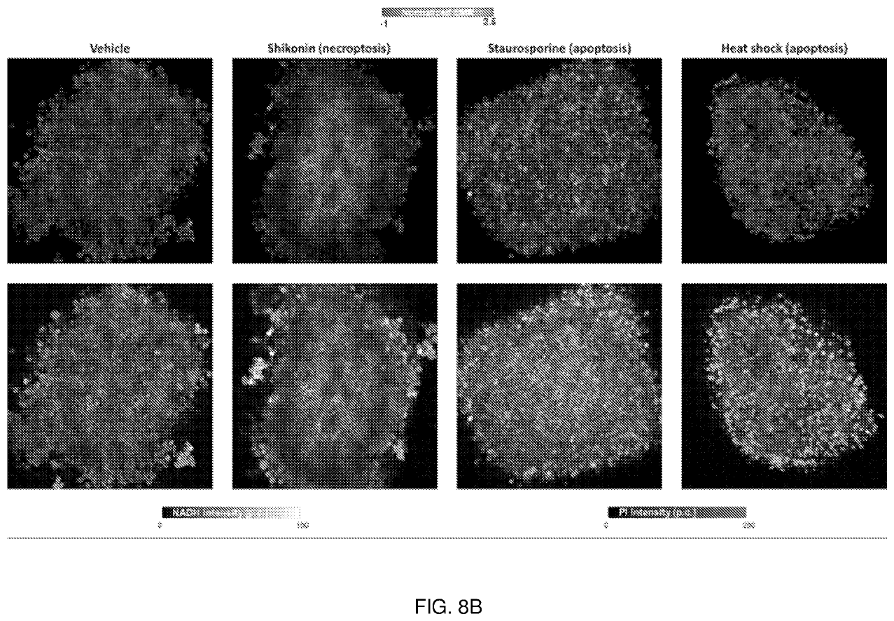

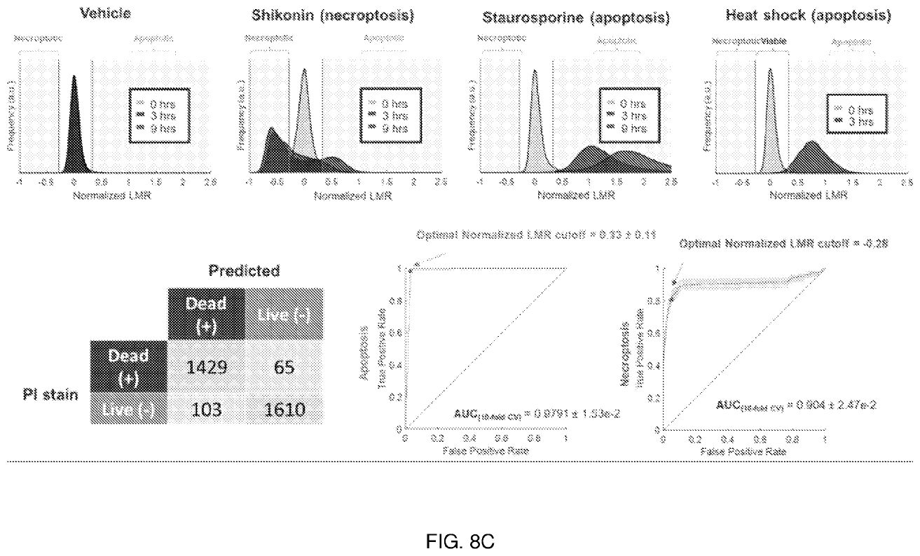

To qualify observed shifts in the LMR, the ground truth nuclear localizing dyes for cleaved caspase 3/7 activity (Invitrogen) or necrosis (propidium iodide) were added. Voronoi cells that stained positive for propidium iodide were marked as dead and those that were not stained with PI were marked as live. Area under the receiver operating characteristic (AUROC) logistic regression (10-fold cross-validation) analysis enabled us to set shift values on the normalized LMR histogram which showed best correlation between LMR and PI staining on Voronoi cells (FIGS. 7 and 8).

Cell death via apoptosis was induced using staurosporine. Apoptosis induced via heat shock also confirmed this data. (FIG. 8). A large, reproducible shift to the right in the LMR was observed relative to time zero healthy tissue. Probe data from Caspase 3/7 and P1 confirmed the shift for LMR values indicating live tissue vs. dead tissue. Cell death via necroptosis was induced using Shikonin. A large, reproducible shift to the left was induced in the LMR relative to healthy tissue at time zero. PI confirmed the necroptosis signature. There was little to no Caspase 3/7 staining in this shikonin-stressed tissue as the principal mode of cell death does not go through effector caspases 3 or 7.

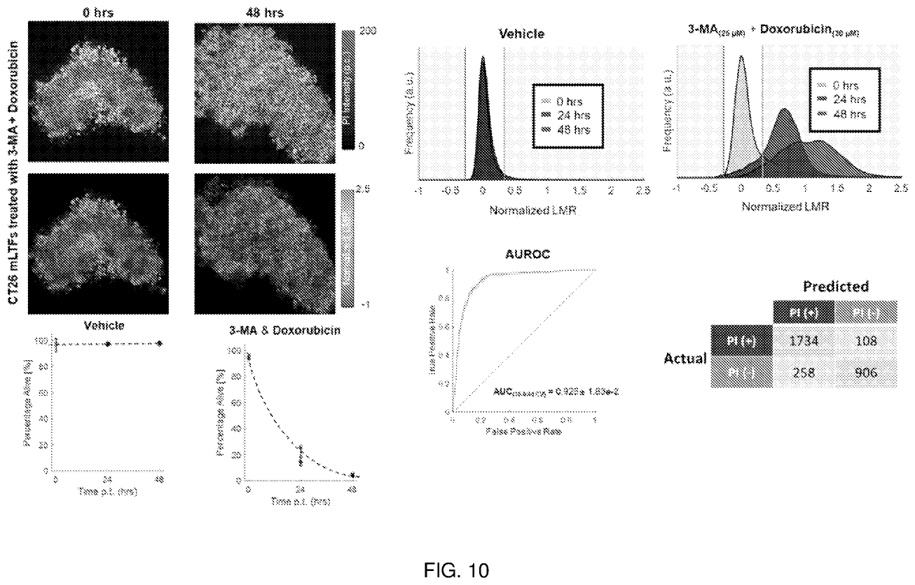

To validate the imaging routine in a model with clinical relevancy, CT26 mLTFs were treated with a combination of meta-methoxyamphetamine (3MA) and doxorubicin over a duration of 48 hours (FIG. 10). For this, mLTFs were imaged every 24 hours following treatment for longitudinal assessment of chemotherapeutic efficacy. Linear metabolic shift values obtained in separate CT26 control experiments were applied to Voronoi segmented images to classify individual cells as alive or dead. Data were plotted in a time course indicating the activity of the drug combination. AUROC analysis indicated that the metabolic shift relative to baseline LMR was robust for following the induced cell death.

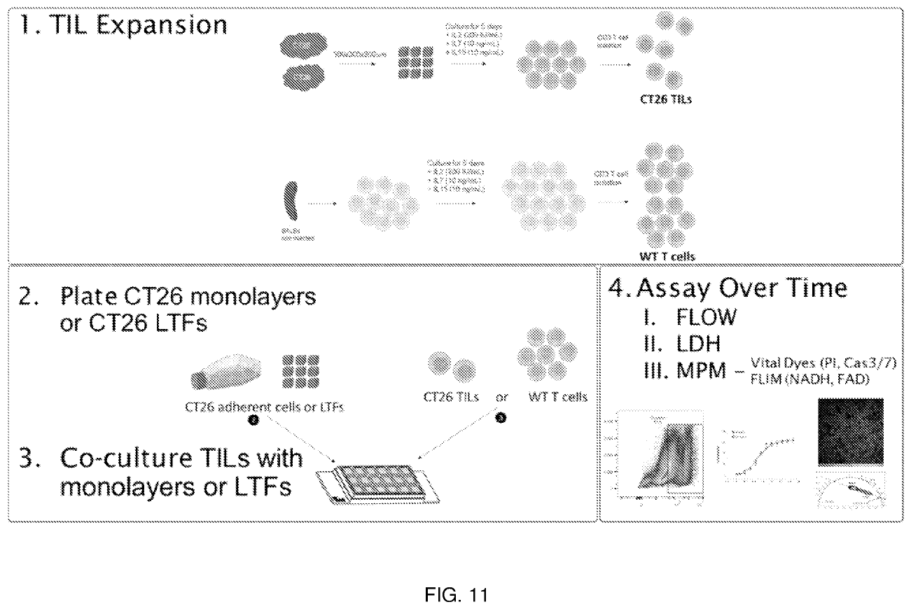

T cell-induced cytotoxicity via granzyme α-induced apoptosis was explored using tumor infiltrating lymphocytes (TILs) isolated from CT26 syngeneic tumors in both CT26 monolayers and CT26 LTFs. TILs were isolated from CT26 fragments using negative selection for T cells. Briefly, CT26 fragments were incubated for 24 h to allow the efflux of TILs into the surrounding media. After the incubation period, the fragments were filtered from the media and the remaining cells in the media were subjected to negative selection for T cells following the standard protocol from Stem Cell Technologies. Isolated CD3+ TILs were either used immediately for multiphoton microscopy (MPM) experiments or activated for 48-72 h with PMA/Ionomycin prior to MPM experiments (FIG. 11).

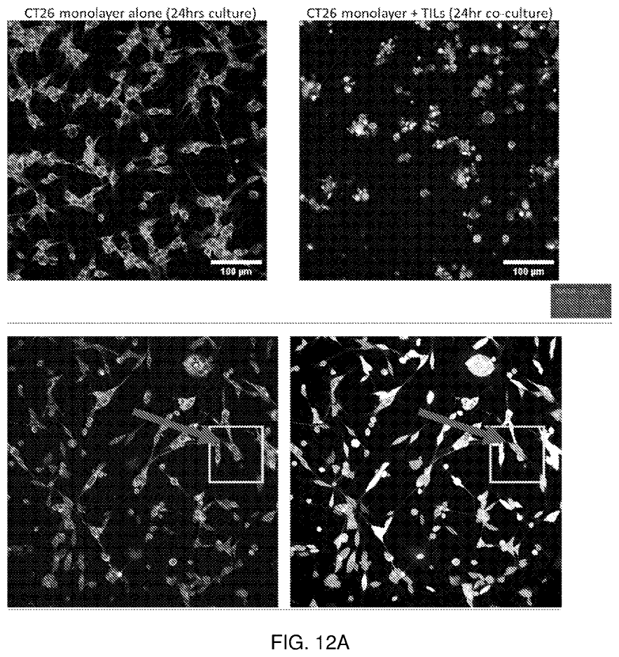

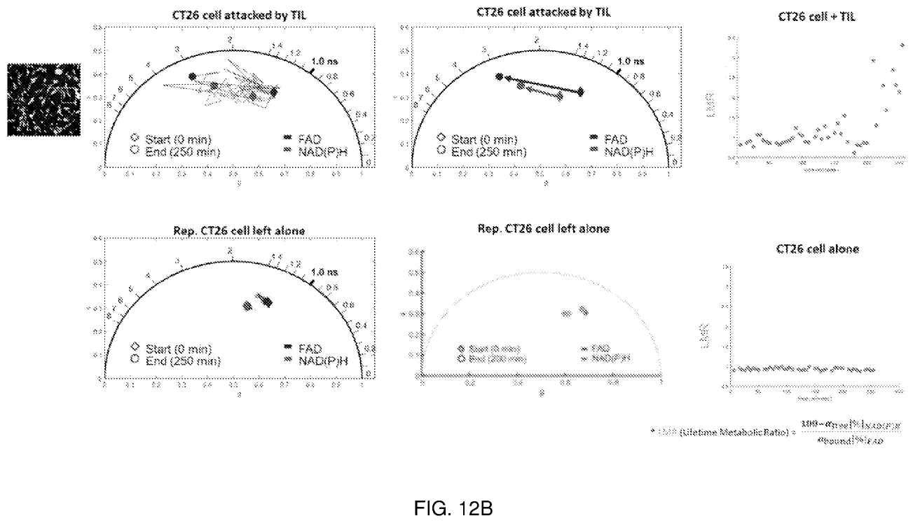

To characterize the metabolic signature of a target tumor cell that encounters an activated TIL, isolated TILs were co-cultured with a monolayer of CT26 cells. TILs were tracked to identify specific points in time when they came into contact with target cells. For those target cells with a contact event, phasor analysis was performed for NAD(P)H and FAD and a normalized LMR shift was calculated to quantify metabolic changes. These signals often preceded cellular blebbing, a known phenotype of cells undergoing apoptosis, and were in excess of the LMR shift value indicative of cell death. Death signatures were further validated using the presence or absence of PI and/or a caspase 3/7 sensor (FIG. 12). Additional experiments were also conducted in CT26 LTFs. Single CT26 LTFs were placed in conical well plates (Insphero) and co-cultured with PMA/Ionomycin-activated TILs. After the addition of TILs, fragments were imaged periodically over a 24-hour period at imaging depths of 50 or 75 microns into the sample. TILs were observed to associate with the periphery of the LTF and then gradually infiltrate the fragment over several hours. After 24 hours, whole tissue changes were observed via phasor plots relative to the control tissue.

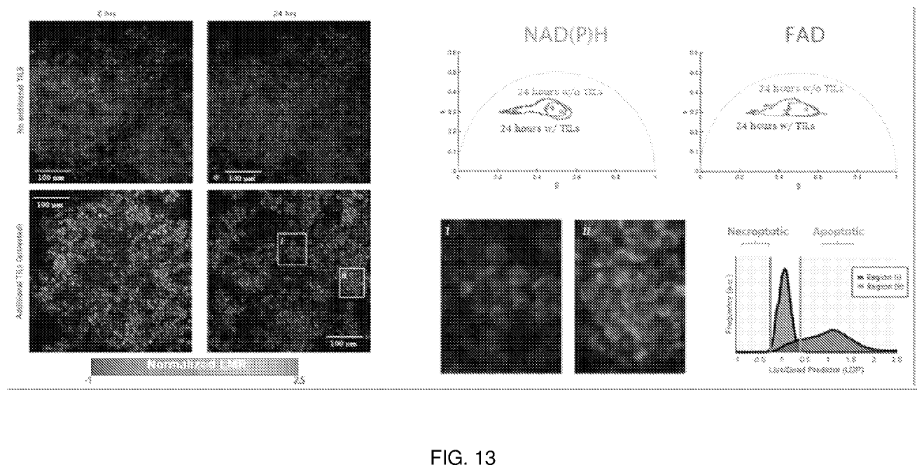

Punctate regions of significantly changed LMR were observed over time. Zooming in on these areas, cell death was indicated by the LMR shift values in 50×50 micron areas (FIG. 13). To provide an orthogonal verification for the observed TIL cytotoxicity, flow cytometry and lactate dehydrogenase (LDH) release assays were performed on the TIL-CT26 co-cultures (FIG. 11).

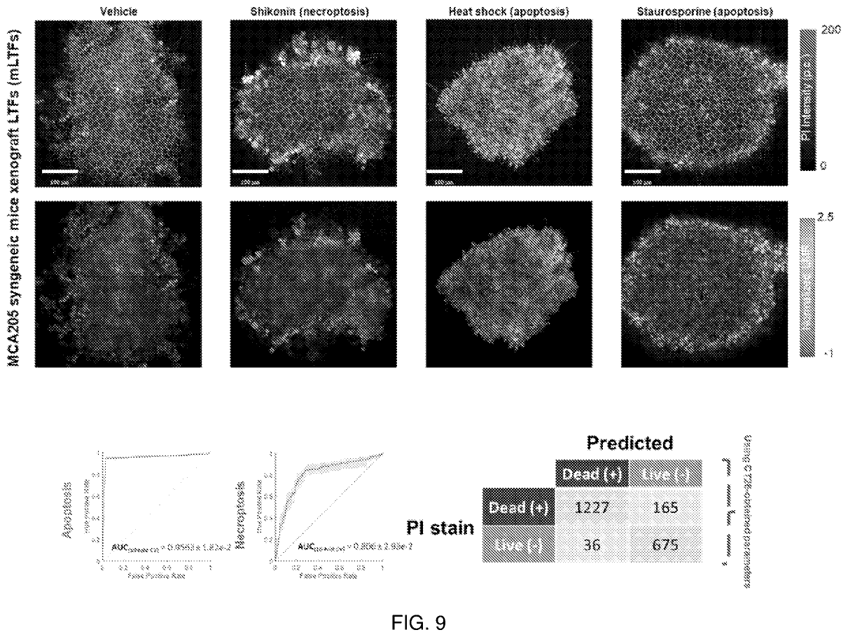

To verify that the observed shifts in the baseline normalized LMR had broader application, data were taken on other syngeneic tumor fragments. MCA205 fragments were imaged and the shift values obtained in CT26 “training” experiments used to segment the resultant images into live and dead fractions (FIG. 9). Groups of fragments were subjected to various treatments to induce known modes of cell death. These modes were confirmed with ground truth dyes indicating apoptosis or necrosis. The ground truth dye propidium iodide was used to test the statistical accuracy of the result. These data confirmed the usefulness of the LMR shift values seen in the CT26 samples. However, the statistical correlation was not as high for apoptotic cell death mechanisms and yielded AUC values of 0.806 vs. 0.90 for test samples of CT26. This was most likely due to greater sample heterogeneity relative to CT26 samples which manifested quantitatively as a broadening of the LMR histogram peaks representing initial and post treatment metabolic states. As a result, a single shift value was less precise in splitting live from dead cell fractions.

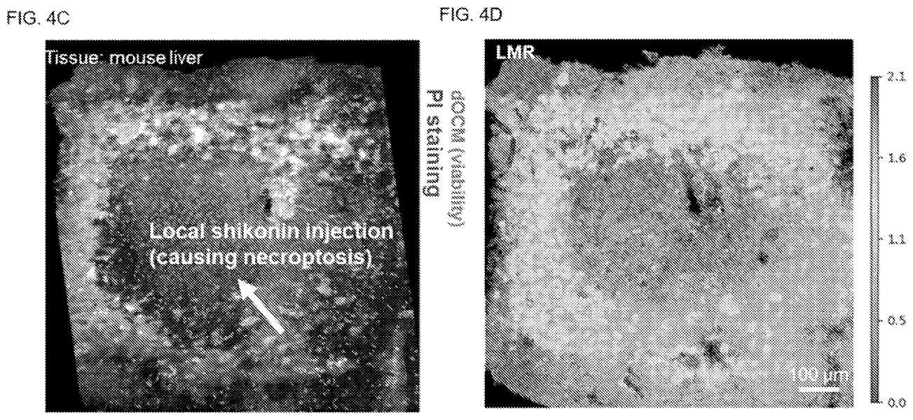

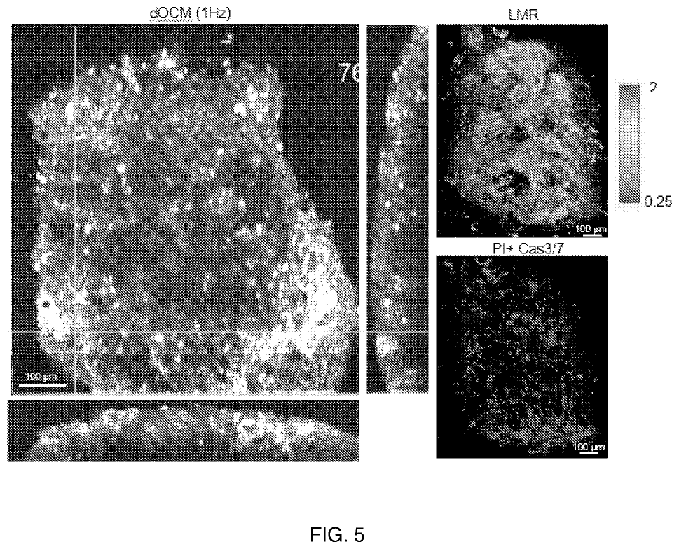

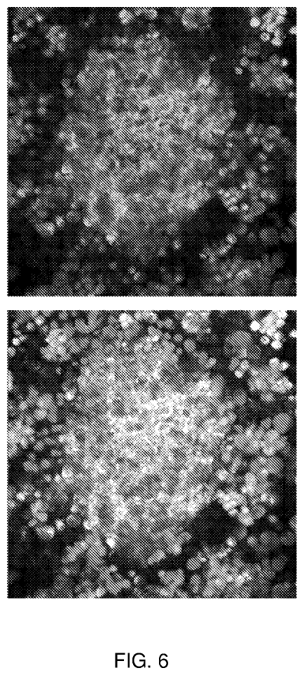

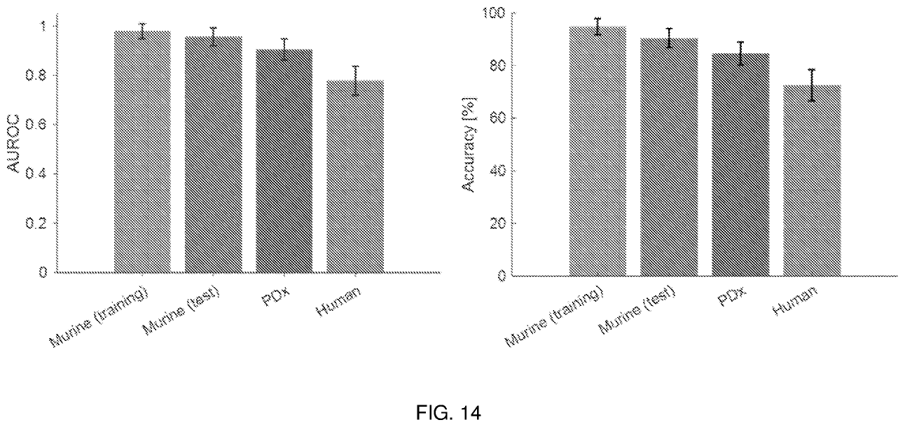

Data taken on increasingly complex (heterogeneous) tissue, which showed greater heterogeneity in starting metabolic state, was obtained and shift analysis performed. LTFs from patient-derived xenografts and from primary human cancer tissue were imaged, the LMR determined, and analyzed via normalized LMR shift analysis relative to an average baseline value for healthy tissue. The data showed directional shifts similar to those in CT26 syngeneic and MCA205 LTFs. However, the statistical correlation declined as a function of starting tissue heterogeneity (FIG. 14). Close analysis (FIG. 4A-4B and FIG. 5) showed areas in heterogeneous tissue which had a baseline healthy metabolic state that was different from adjacent cells leading us to conclude that the normalized baseline value had certain limitations in complex tissue.

FIG. 4C-D shows multimodal imaging of tissue viability with a piece of mouse liver using dOCM and FLIM. 100 nL shikonin (6 μM) was injected into the center of the tissue to create necroptosis indicted by the white arrow. Scale bar: 100 μm. FIG. 4C shows composite image of dOCM indicating variable cells (green) and dead tissue stained by propidium iodide (magenta). FIG. 4D shows lifetime metabolic ratio (LMR) measured by FLIM from the same tissue. LMR values are indicated by the color bar. Viable cells exhibit higher LMR values compared to dead cells.

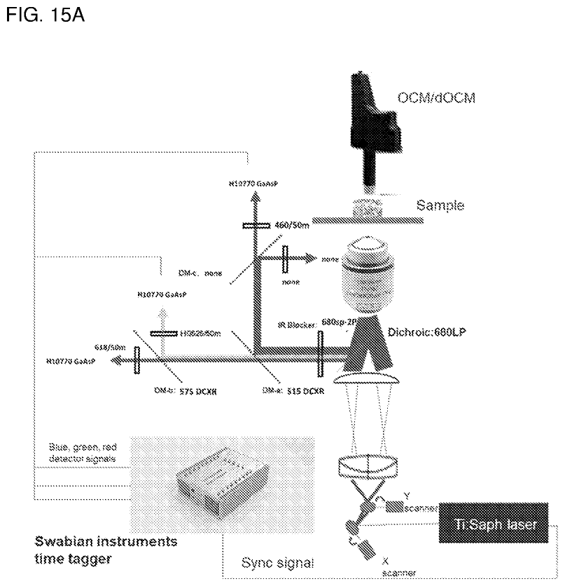

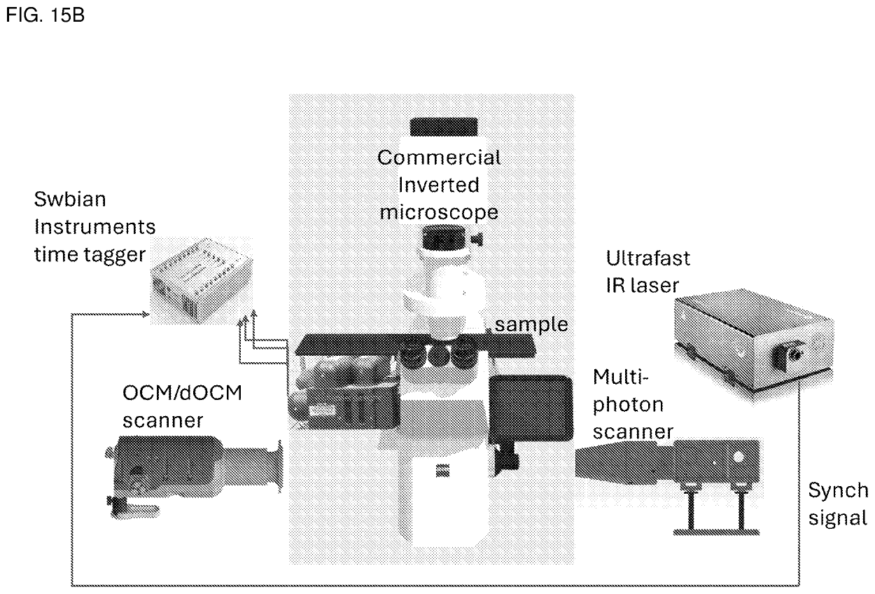

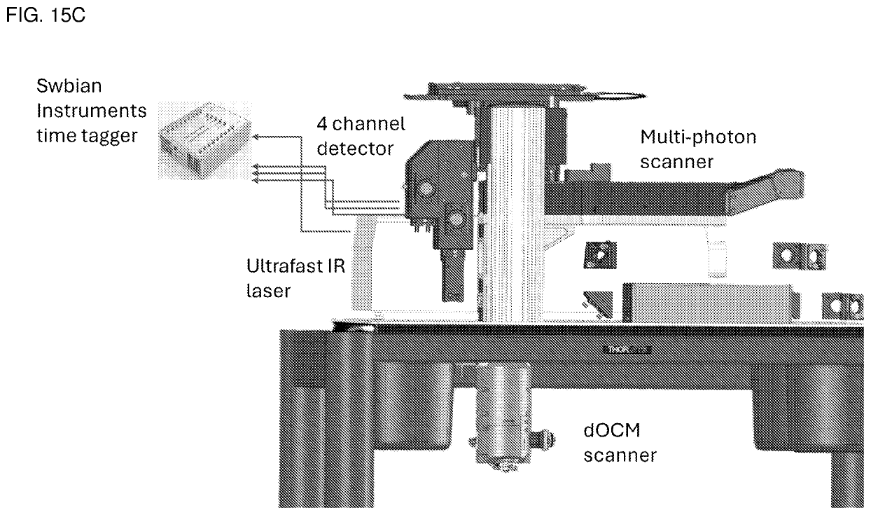

A need was identified in complex heterogeneous tissue to set healthy baseline metabolic state to effectively use normalized LMR shift values and assay cell death mechanisms. To address this, an instrument was constructed which allowed the simultaneous OCM, dOCM, and multi-channel MPM FLIM data to be taken in a manner that was co-registered in 3D space (FIG. 15A). For each pixel in the time series of OCM intensity I(x, y, z, t), fast Fourier transform was performed to obtain the power spectrum P(x, y, z, f). The amplitude of the power spectrum between 0.14 Hz-1 Hz was averaged to obtain the dOCM contrasts for live cells. In this frequency band, dead tissues shows significantly lower amplitudes in P(x, y, z, f) compared to live tissues. This allows us to spatially localized LMR values for healthy tissue.

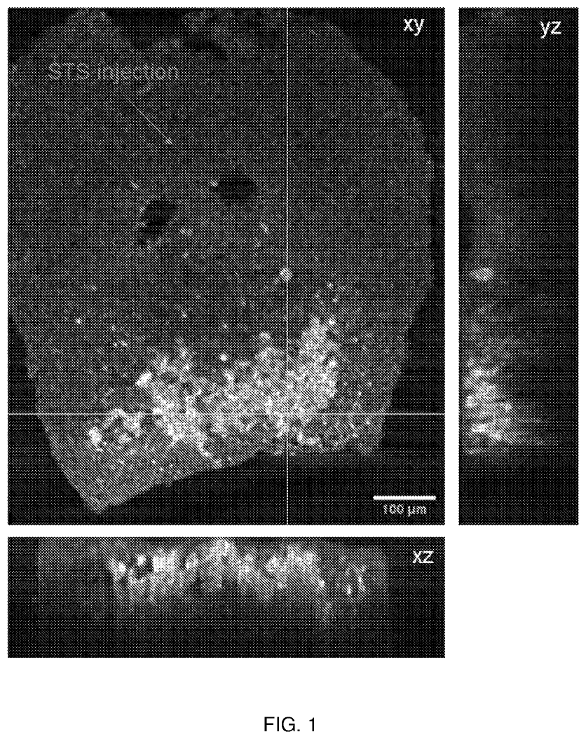

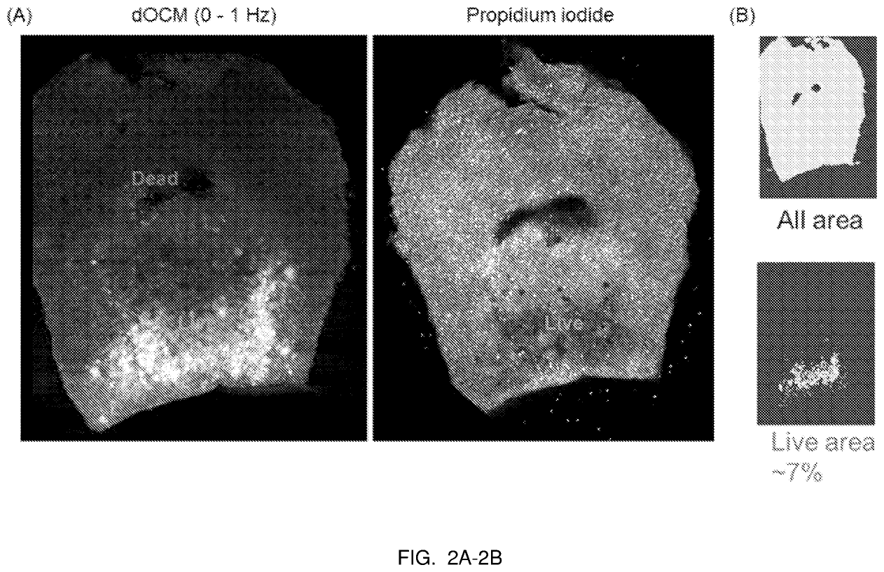

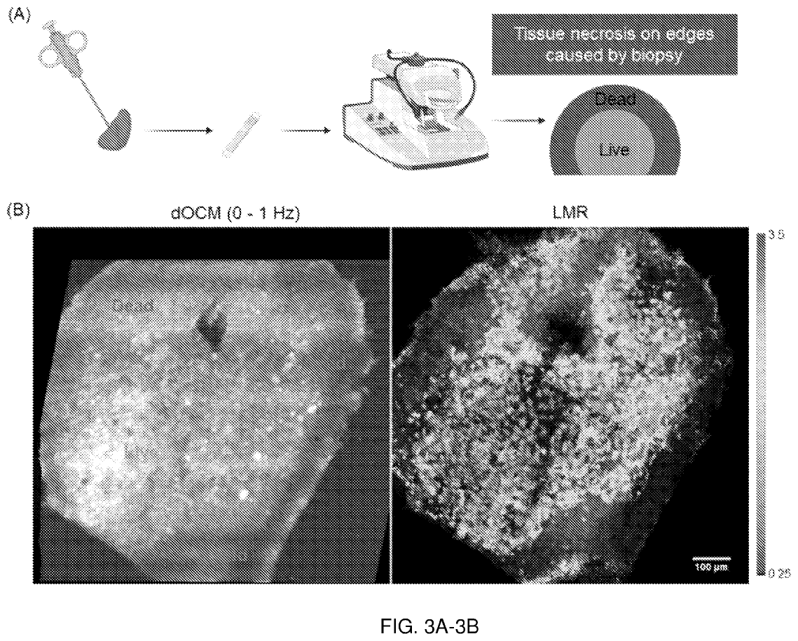

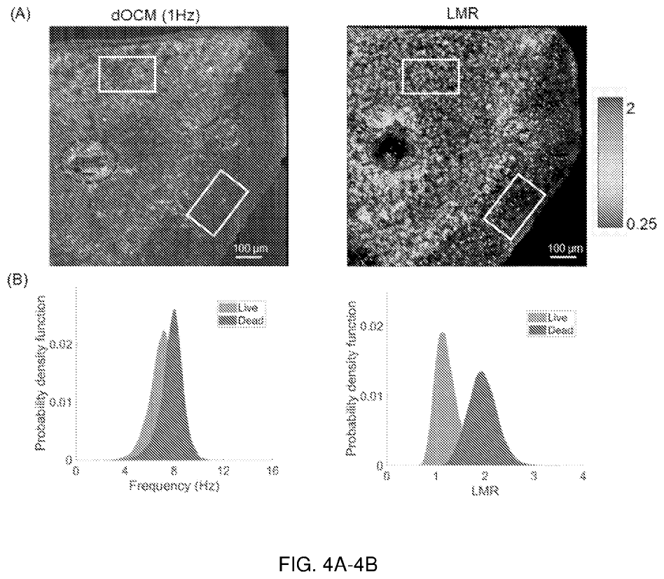

A 300 μm thickness mouse liver slice was used to demonstrate the capability of dOCM to identify live cells (FIG. 1). Local injection of 200 nL staurosporine (STS, 2 μM in DMSO) caused tissue death in a portion of the sample. Live cells were visible in the dOCM image volume. The live cells showed high contrast compared to dead regions. Propidium Iodide was added and imaged using MPM to verify the dead regions of the tissue which showed an opposite staining pattern relative to the region visible with the dOCM (FIG. 2), dOCM signatures and LMR signatures from multi-photon FLIM along with propidium iodide were taken on the same tissue to show colocalization of live and dead signals. Good correlation is observed between the dOCM signal and the expected LMR signatures in this monolithic tissue (FIG. 3). However, certain segments of images taken in tissue with various cell types represented did not correlate well (FIG. 4-5). In those areas, the dOCM is used to establish a ground truth for living tissue that can in turn be used to qualify the LMR and set specific discreet baseline LMR values for living tissue in these regions. This is as opposed to using a generic, specific value determined with an assumption that the tissue is monolithic.

The methods described herein utilize variety of imaging techniques to produce data which are segmented and analyzed to characterize the viability of cells in a biological sample. The methods described herein further elucidate the particular mechanism by which cells in the field of view lose viability (e.g., necroptosis or apoptosis).

### Example 2

This example describes schematics of constructed microscope embodiments allowing for simultaneous visualization of a sample by OCM/dOCM and multi-channel multi-photon microscopy.

FIG. 15A shows a system configuration that can employ OCM/dOCM from the top of the sample while either sequentially or simultaneously employ multi-photon imaging from the bottom of the sample. Also depicted are the scanners for the multiphoton, the time tagger for FLIM and the optics for separating out signals. The addition of the Swabian time tagger allows for multi-channel FLIM measurements described in this document.

The configuration shown in FIG. 15B brings the multi-photon scanner in from one side of the microscope (right side depicted here) and the OCM/dOCM scanner from the other side of the microscope. This configuration is suitable for sequential imaging of the sample with OCM/dOCM and multi-photon imaging, FLIM.

FIG. 15C shows a configuration which mounts the multi-photon scanner for imaging and FLIM from the right side of the microscope and the OCM/dOCM scanner from the bottom of the microscope. This configuration is suitable for either simultaneous multi-photon imaging and FLIM with OCM/dOCM or sequential imaging of the acquisition modes.

### Example 3

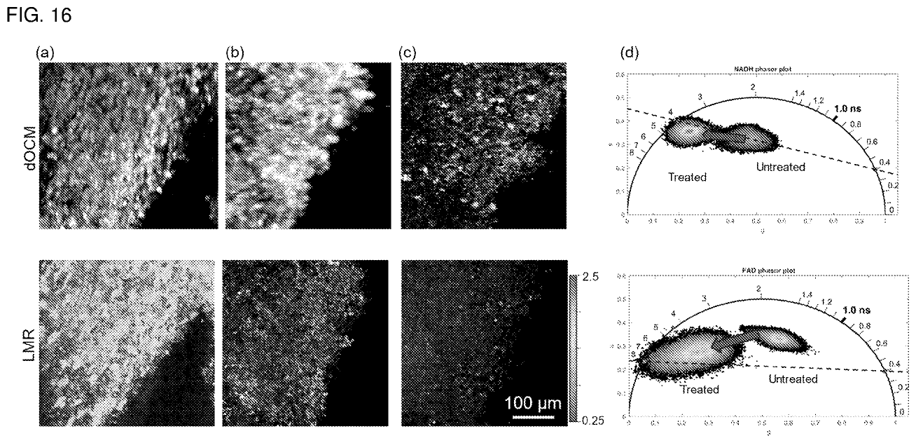

FIG. 16 presents multimodal imaging of tissue viability with primary human lung cancer tissue using combined dOCM and FLIM. (a) dOCM and LMR images of a human lung cancer tissue slice. (b) dOCM and LMR images of another lung tissue slice from the same sample. The metabolic state of this sample is different from the tissue slice in (a). (c) The tissue slice in (a) was treated with 10 μM staurosporine. The slice was imaged 24 hours after treatment. Less viable cells were revealed by dOCM; the metabolic state was shifted compared to that prior to treatment. (d) The shift of metabolism was visualized by the NADH and FAD phasor plots indicating sample undergoing apoptosis, consistent with FIG. 8C. Scale bar 100 μm.

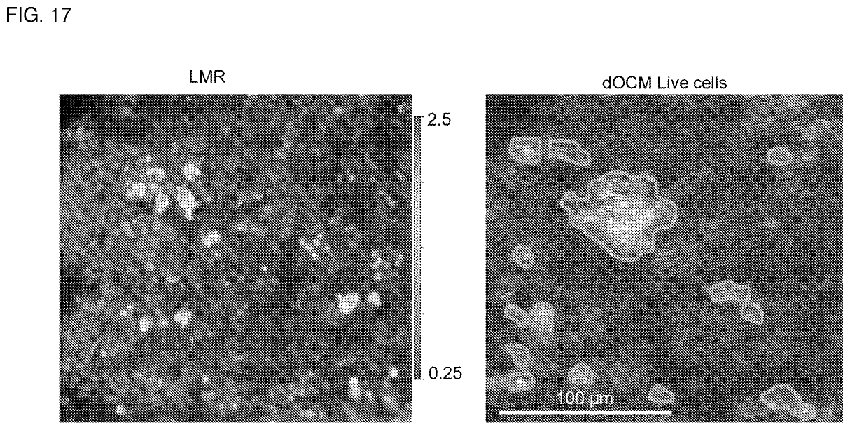

FIG. 17 presents multimodal imaging of primary human ovary cancer tissue with low viability using combined dOCM and FLIM. Live cells are revealed by the dOCM. The live cells showed different metabolic states (LMR) compared with the dead cells. Moreover, the metabolic states were different from the sample in FIG. 16 without treatment. Scale bar: 100 μm.

1. A method of evaluating a biological sample, comprising
a) imaging the biological sample with OCM/dOCM (e.g., OCM and/or dOCM) to identify a live cell fraction in the biological sample; and
b) imaging the biological sample with FLIM to classify the cell state within the identified live cell fraction.

2. The method of claim 1, wherein the imaging the biological sample with OCM/dOCM to identify a live cell fraction in the biological sample and the imaging the biological sample with FLIM to classify the cell state is contemporaneous or nearly contemporaneous.

3. The method of claim 1, wherein the biological sample is one or more selected from a living tissue sample, an organ sample, a whole blood sample, a plasma sample, a serum sample, and a lavage sample.

4. The method of claim 3, wherein the living tissue sample is selected from one or more of:
whole biopsies or biopsies that have been cut longitudinally into one or more strips,
tissue coins cut from a tissue biopsy,
a whole biopsy,
a bisected biopsy tissue sample, and
a living tissue fragment.

5. The method of claim 1, wherein the biological sample comprises cytotoxic T-cell lymphocytes (CTLs).

6. The method of claim 1, wherein classifying the cell state with FLIM comprises classifying the metabolic state and/or the metabolic signature in the identified live cell fraction.

7. The method of claim 6, wherein the FLIM comprises multi-photon FLIM (MP-FLIM).

8. The method of claim 7, wherein MP-FLIM provides a signal from a first intrinsic fluorophore and a signal from a second intrinsic fluorophore, wherein the first intrinsic fluorophore is NAD(P)H and/or the second intrinsic fluorophore is FAD.

9. The method of claim 8, wherein classifying cell state comprises calculating a lifetime metabolic ratio (LMR) of the identified live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD.

10. The method of claim 8, wherein the signal from the first intrinsic fluorophore and the signal from the second intrinsic fluorophore are identified by phasor segmentation analysis.

11. The method of claim 8, wherein the signal from the first intrinsic fluorophore and the signal from the second intrinsic fluorophore are identified by phasor segmentation analysis.

12. The method of claim 8, wherein the signal from the first intrinsic fluorophore corresponds to an amount of free NAD(P)H and wherein the signal from the second intrinsic fluorophore corresponds to an amount of enzyme-bound FAD.

13. The method of claim 12, wherein an increased signal from the first intrinsic fluorophore relative to a threshold value and/or an increased signal from the second intrinsic fluorophore relative to a threshold value indicates a glycolytic metabolic state in the biological sample.

14. The method of claim 1, wherein the biological sample is further evaluated with one or more of the following additional imaging modalities: mass spectrometry (MS), nuclear magnetic resonance spectroscopy, magnetic resonance imaging, light sheet microscopy, lattice light sheet microscopy, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography), SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonics generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

15. The method of claim 1, further comprising:
c) contacting the biological sample with one or more potential therapeutic agents, wherein the contacting is subsequent to the imaging of the biological sample with OCM/dOCM and FLIM; and
d) imaging the biological sample with OCM/dOCM and/or FLIM to re-classify the cell state within the identified live cell fraction to measure classification differences pre- and post-contacting of the biological sample with the one or more potential therapeutic agents.

16. The method of claim 15, wherein measuring the classification differences comprises measuring a metabolic shift of the identified and classified live cell fraction from a baseline metabolic state.

17. The method of claim 16, wherein the baseline metabolic state is determined prior to contacting the identified and classified live cell fraction with the potential therapeutic agent.

18. The method of claim 15, wherein the one or more potential therapeutic agents is selected from one or more of a small molecule, a polypeptide or peptide fragment, an siRNA, a cellular therapy (e.g., tumor-infiltrating lymphocyte therapy, CAR T-cell therapy, T-cell receptor-based therapy, natural killer cell therapy, etc.), an oncolytic virus, a metabolite treatment (e.g., an adenosine treatment), or an antibody or fragment thereof.

19. An ex vivo method of assessing one or more potential therapeutic agents, comprising
a) imaging a biological sample with OCM/dOCM to identify a live cell fraction in the biological sample;
b) imaging the biological sample with FLIM to classify the cell state within the biological;
c) contacting the biological sample with one or more potential therapeutic agents, wherein the contacting is subsequent to the imaging of the biological sample with OCM/dOCM and FLIM; and
d) imaging the biological sample with OCM/dOCM and/or FLIM to re-classify the cell state within the biological sample to measure classification differences pre- and post-contacting of the biological sample with the one or more potential therapeutic agents.

20. The method of claim 19, wherein each assessment is contemporaneous or nearly-contemporaneous.

21. The method of claim 19, wherein the biological sample is one or more selected from a living tissue sample, an organ sample, a whole blood sample, a plasma sample, a serum sample, and a lavage sample.

22. The method of claim 21, wherein the living tissue sample is selected from one or more of:
whole biopsies or biopsies that have been cut longitudinally into one or more strips,
tissue coins cut from a tissue biopsy,
a whole biopsy,
a bisected biopsy tissue sample, and
a living tissue fragment.

23. The method of claim 21, wherein the biological sample comprises CTLs.

24. The method of claim 21, wherein classifying the cell state with FLIM comprises classifying the metabolic state.

25. The method of claim 24, wherein the FLIM comprises multi-photon FLIM (MP-FLIM).

26. The method of claim 25, wherein MP-FLIM provides a signal from a first intrinsic fluorophore and a signal from a second intrinsic fluorophore, wherein the first intrinsic fluorophore is NAD(P)H and/or the second intrinsic fluorophore is FAD.

27. The method of claim 26, wherein the signal from the first intrinsic fluorophore and the signal from the second intrinsic fluorophore are identified by phasor segmentation analysis.

28. The method of claim 26, wherein the signal from the first intrinsic fluorophore corresponds to an amount of free NAD(P)H and wherein the signal from the second intrinsic fluorophore corresponds to an amount of enzyme-bound FAD.

29. The method of claim 19, wherein the assessment differences pre- and post-contacting of the biological sample with the one or more potential therapeutic agents comprises measuring a metabolic shift of the biological sample from a baseline metabolic state.

30. The method of claim 29, wherein the baseline metabolic state is determined prior to contacting the biological sample with the potential therapeutic agent.

31. The method of claim 19, wherein the one or more potential therapeutic agents is selected from one or more of a small molecule, a polypeptide or peptide fragment, an siRNA, a cellular therapy, an oncolytic virus, a metabolite treatment (e.g., an adenosine treatment), or an antibody or fragment thereof.

32. The method of claim 19, wherein the cellular therapy is one or more selected from tumor-infiltrating lymphocyte therapy, CAR T-cell therapy, T-cell receptor-based therapy, and natural killer cell therapy.

33. An ex vivo method of assessing a biological sample, comprising:
providing
a biological sample, and
a processor comprising software that, when executed, causes the processor to manually or automatically:
image the biological sample with OCM and/or dOCM to identify a live cell fraction in the biological sample; and
image the biological sample with FLIM to classify the cell state within the identified live cell fraction; and
provide a report to a user of the identified and classified live cell fraction;
executing the processor to
image the biological sample with OCM/dOCM to identify a live cell fraction in the biological sample; and
image the biological sample with FLIM to classify the cell state within the identified live cell fraction, and
provide a report to a user of the identified and classified live cell fraction.

34. The method of claim 33, wherein the imaging the biological sample with OCM and/or dOCM to identify a live cell fraction in the biological sample and the imaging the biological sample with FLIM to classify the cell state within the biological sample is contemporaneous or nearly contemporaneous.

35. The method of claim 33, wherein the biological sample is one or more selected from a living tissue sample, an organ sample, a whole blood sample, a plasma sample, a serum sample, and a lavage sample.

36. The method of claim 35, wherein the living tissue sample is selected from one or more of:
whole biopsies or biopsies that have been cut longitudinally into one or more strips,
tissue coins cut from a tissue biopsy,
a whole biopsy,
a bisected biopsy tissue sample, and
a living tissue fragment.

37. The method of claim 33, wherein the biological sample comprises CTLs.

38. The method of claim 33, wherein classifying the cell state with FLIM comprises classifying the metabolic state in the biological sample.

39. The method of claim 38, wherein the FLIM comprises multi-photon FLIM (MP-FLIM).

40. The method of claim 39, wherein MP-FLIM provides a signal from a first intrinsic fluorophore and a signal from a second intrinsic fluorophore, wherein the first intrinsic fluorophore is NAD(P)H and/or the second intrinsic fluorophore is FAD.

41. The method of claim 39, wherein the signal from the first intrinsic fluorophore and the signal from the second intrinsic fluorophore are identified by phasor segmentation analysis.

42. The method of claim 39, wherein the signal from the first intrinsic fluorophore and the signal from the second intrinsic fluorophore are identified by phasor segmentation analysis.

43. The method of claim 39, wherein the signal from the first intrinsic fluorophore corresponds to an amount of free NAD(P)H and wherein the signal from the second intrinsic fluorophore corresponds to an amount of enzyme-bound FAD.

44. The method of claim 43, wherein an increased signal from the first intrinsic fluorophore relative to a threshold value and/or an increased signal from the second intrinsic fluorophore relative to a threshold value indicates a glycolytic metabolic state in the biological sample.

45. The method of claim 39, wherein classifying cell state comprises calculating a lifetime metabolic ratio (LMR) of the biological sample, wherein the LMR is calculated by dividing a signal from the first intrinsic fluorophore corresponding to the amount of enzyme-bound NAD by a signal from the second intrinsic fluorophore corresponding to the amount of enzyme-bound FAD.

46. The method of claim 33, wherein the biological sample is further evaluated with one or more of the following additional imaging modalities: mass spectrometry (MS), nuclear magnetic resonance spectroscopy, magnetic resonance imaging, light sheet microscopy, lattice light sheet microscopy, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography), SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonics generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

47. The method of claim 33, wherein the processor further comprises a display configured to display the provided report.

48. An ex vivo method of assessing one or more potential therapeutic agents, comprising providing:
a biological sample, and
a processor comprising software that, when executed, causes the processor to manually or automatically:
image the biological sample with OCM/dOCM to identify a live cell fraction in the biological sample;
image the biological sample with FLIM to classify the cell state within the biological sample;
contact the biological sample with one or more potential therapeutic agents subsequent to the imaging of the biological sample with OCM/dOCM and FLIM;
image the biological sample with OCM/dOCM and/or FLIM to re-classify the cell state within the biological sample;
measure classification differences pre- and post-contacting of the biological sample with the one or more potential therapeutic agents;
provide a report to a user of one or more of:
an identified and classified live cell fraction;
a re-classified identified and classified live cell fraction; and
classification differences pre- and post-contacting of the biological sample with the one or more potential therapeutic agents;
executing the software to:
image the biological sample with OCM/dOCM to identify a live cell fraction in the biological sample;
image the biological sample with FLIM to classify the cell state within the biological sample;
contact the biological sample with one or more potential therapeutic agents subsequent to the imaging of the biological sample with OCM/dOCM and FLIM;
image the biological sample with OCM/dOCM and/or FLIM to re-classify the cell state within the biological sample;
measure classification differences pre- and post-contacting of the biological sample with the one or more potential therapeutic agents;
provide a report to a user of one or more of:
an identified and classified live cell fraction;
a re-classified identified and classified live cell fraction; and
classification differences pre- and post-contacting of the biological sample with the one or more potential therapeutic agents.

49. The method of claim 48, wherein the imaging the biological sample with OCM/dOCM (e.g., OCM and/or dOCM) to identify a live cell fraction in the biological sample and the imaging the biological sample with FLIM to classify the cell state within the identified live cell fraction is contemporaneous or nearly contemporaneous.

50. The method of claim 48, wherein the biological sample is one or more selected from:
a living tissue sample, an organ sample, a whole blood sample, a plasma sample, a serum sample, and a lavage sample.

51. The method of claim 50, wherein the living tissue sample is selected from one or more of:
whole biopsies or biopsies that have been cut longitudinally into one or more strips,
tissue coins cut from a tissue biopsy,
a whole biopsy,
a bisected biopsy tissue sample, and
a living tissue fragment.

52. The method of claim 48, wherein the one or more potential therapeutic agents is selected from one or more of a small molecule, a polypeptide or peptide fragment, an siRNA, a cellular therapy (e.g., tumor-infiltrating lymphocyte therapy, CAR T-cell therapy, T-cell receptor-based therapy, natural killer cell therapy, etc.), an oncolytic virus, a metabolite treatment (e.g., an adenosine treatment), or an antibody or fragment thereof.

53. The method of claim 48, wherein classifying the cell state with FLIM comprises classifying the metabolic state in the biological sample.

54. The method of claim 53, wherein the FLIM comprises multi-photon FLIM (MP-FLIM).

55. The method of claim 54, wherein MP-FLIM provides a signal from a first intrinsic fluorophore and a signal from a second intrinsic fluorophore, wherein the first intrinsic fluorophore is NAD(P)H and/or the second intrinsic fluorophore is FAD.

56. The method of claim 54, wherein the signal from the first intrinsic fluorophore and the signal from the second intrinsic fluorophore are identified by phasor segmentation analysis.

57. The method of claim 54, wherein the signal from the first intrinsic fluorophore and the signal from the second intrinsic fluorophore are identified by phasor segmentation analysis.

58. The method of claim 54, wherein the signal from the first intrinsic fluorophore corresponds to an amount of free NAD(P)H and wherein the signal from the second intrinsic fluorophore corresponds to an amount of enzyme-bound FAD.

59. The method of claim 48, wherein an increased signal from the first intrinsic fluorophore relative to a threshold value and/or an increased signal from the second intrinsic fluorophore relative to a threshold value indicates a glycolytic metabolic state in the biological sample.

60. The method of claim 48, wherein classifying cell state comprises calculating a lifetime metabolic ratio (LMR) of the biological sample, wherein the LMR is calculated by dividing a signal from the first intrinsic fluorophore corresponding to the amount of enzyme-bound NAD by a signal from the second intrinsic fluorophore corresponding to the amount of enzyme-bound FAD.

61. The method of claim 48, wherein the biological sample is further evaluated with one or more of the following additional imaging modalities: mass spectrometry (MS), nuclear magnetic resonance spectroscopy, magnetic resonance imaging, light sheet microscopy, lattice light sheet microscopy, proton magnetic resonance spectroscopy, holographic microscopy, interference phase microscopy, quantitative phase microscopy, polarized phase microscopy, contrast phase microscopy, time-lapse imaging microscopy, surface enhanced Raman spectroscopy, videography, manual visual analysis, automated visual analysis, traction force microscopy, optical coherent tomography (OCT), intravascular ultra sound (IVUS), photoacoustics (PA), near-infra-red spectroscopy, impedance spectroscopy, SLOT (scanning laser optical tomography), SPIM (single plane illumination microscopy), optical projection tomography (OPT), wide-field microscopy, transmission microscopy, confocal fluorescence microscopy, coherent anti-Stokes Raman scattering (CARS), nonlinear microscopy (e.g., two or three photon microscopy or microscopy using high harmonics generation (HHG)), confocal theta microscopy, stimulated emission detection microscopy (STED), structured illumination microscopy (SIM), localization microscopy (PALM/STORM), x-ray microscopy, x-ray tomography, an imaging ultrasound method, radioprobes, or combinations thereof.

62. The method of claim 48, wherein the processor further comprises a display configured to display the provided report.

63. A system comprising:
a biological sample, and
a processor comprising software that, when executed, causes the processor to manually or automatically:
image the biological sample with OCM/dOCM (e.g., OCM and/or dOCM) to identify a live cell fraction in the biological sample; and
image the biological sample with FLIM to classify the cell state within the biological sample; and
provide a report to a user of the identified and classified live cell fraction and/or biological sample.

64. A system comprising:
a biological sample, and
a processor comprising software that, when executed, causes the processor to manually or automatically:
image the biological sample with OCM/dOCM (e.g., OCM and/or dOCM) to identify a live cell fraction in the biological sample;
image the biological sample with FLIM to classify the cell state within the biological sample;
contact the biological sample with one or more potential therapeutic agents subsequent to the imaging of the biological sample with OCM/dOCM and FLIM;
image the biological sample with OCM/dOCM and/or FLIM to re-classify the cell state within the biological sample;
measure classification differences pre- and post-contacting of the biological sample with the one or more potential therapeutic agents;
provide a report to a user of one or more of:
an identified and classified live cell fraction;
a re-classified identified and classified live cell fraction;
classification differences pre- and post-contacting of the biological sample with the one or more potential therapeutic agents; and
classification differences pre- and post-contacting of identified and classified live cell fraction with the one or more potential therapeutic agents.

65. A method of evaluating a biological sample, the method comprising:
a) imaging a biological sample with optical coherence microscopy (OCM) and/or dynamic optical coherence microscopy (dOCM), thereby identifying a live cell fraction in the biological sample; and
b) imaging the biological sample with fluorescence lifetime imaging microscopy (FLIM) to classify cell state within the live cell fraction of the biological sample.

66. The method of claim 65, wherein FLIM comprises multi-channel FLIM.

67. The method of claim 66, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

68. The method of claim 67, wherein the signal from the first intrinsic fluorophore and the signal from the second intrinsic fluorophore are identified by phasor segmentation analysis.

69. The method of claim 67, wherein classifying cell state comprises calculating a lifetime metabolic ratio (LMR) of the live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD.

70. A method of evaluating a biological sample, the method comprising:
a) imaging a biological sample with optical coherence microscopy (OCM) and/or dynamic optical coherence microscopy (dOCM), thereby identifying a live cell fraction of the biological sample; and
b) imaging the biological sample with fluorescence lifetime imaging microscopy (FLIM) to determine a baseline metabolic signature for the live cell fraction of the biological sample.

71. The method of claim 70, wherein FLIM comprises multi-channel FLIM.

72. The method of claim 71, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

73. The method of claim 72, wherein the signal from the first intrinsic fluorophore and the signal from the second intrinsic fluorophore are identified by phasor segmentation analysis.

74. The method of claim 72, wherein the baseline metabolic signature is determined by calculating a lifetime metabolic ratio (LMR) of the live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD.

75. A method of evaluating a potential therapeutic agent, the method comprising:
a) imaging a biological sample with optical coherence microscopy (OCM) and/or dynamic optical coherence microscopy (dOCM), thereby identifying a live cell fraction of a biological sample;
b) contacting the biological sample with a potential therapeutic agent;
c) measuring a metabolic shift in the live cell fraction from a baseline metabolic signature for the live cell fraction; and
d) classifying cell state within the live cell fraction based upon the metabolic shift measured in the live cell fraction.

76. The method of claim 75, wherein the baseline metabolic signature for the live cell fraction is determined by imaging the biological sample with fluorescence lifetime imaging microscopy (FLIM).

77. The method of claim 76, wherein FLIM comprises multi-channel FLIM.

78. The method of claim 77, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

79. The method of claim 78, wherein the signal from the first intrinsic fluorophore and the signal from the second intrinsic fluorophore are identified by phasor segmentation analysis.

80. The method of claim 78, wherein the baseline metabolic signature is determined by calculating a lifetime metabolic ratio (LMR) of the live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD.

81. The method of claim 75, wherein the metabolic shift in the live cell fraction is measured by FLIM.

82. The method of claim 75, wherein the metabolic shift in the live cell fraction is measured by multi-channel FLIM.

83. The method of claim 82, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

84. The method of claim 83, wherein the metabolic shift in the live cell fraction is determined by calculating a lifetime metabolic ratio (LMR) of the live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD.

85. The method of claim 84, wherein the signal from the first intrinsic fluorophore and the signal from the second intrinsic fluorophore are identified by phasor segmentation analysis.

86. The method of claim 75, wherein a metabolic shift above a threshold value is indicative of cell death.

87. The method of claim 75, wherein a metabolic shift within a first range of reference values is indicative of apoptosis, and/or wherein a metabolic shift within a second range of reference values is indicative of necrosis.

88. The method of claim 75, wherein the potential therapeutic agent comprises a cell.

89. The method of claim 88, wherein the cell comprises a leukocyte.

90. The method of claim 89, wherein the cell comprises a T-cell, a B-cell, an NK cell, or a macrophage.

91. The method of claim 75, wherein the potential therapeutic agent comprises a drug.

92. The method of claim 91, wherein the drug comprises a potential anti-cancer agent.

93. The method of claim 75, wherein the biological sample comprises a tissue sample.

94. The method of claim 93, wherein the tissue sample comprises a tumor tissue sample.

95. A method of evaluating a biological sample, the method comprising:
a) contacting a biological sample with a potential therapeutic agent, wherein the potential therapeutic agent comprises a leukocyte; and
b) imaging the biological sample with fluorescence lifetime imaging microscopy (FLIM) to classify cell state in the biological sample.

96. The method of claim 95, wherein FLIM comprises multi-channel FLIM.

97. The method of claim 96, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

98. The method of claim 97, wherein the signal from the first intrinsic fluorophore and the signal from the second intrinsic fluorophore are identified by phasor segmentation analysis.

99. The method of claim 97, wherein classifying cell state comprises calculating a lifetime metabolic ratio (LMR) of the live cell fraction, wherein the LMR is calculated by dividing a signal from the first intrinsic fluorophore corresponding to the amount of enzyme-bound NAD by a signal from the second intrinsic fluorophore corresponding to the amount of enzyme-bound FAD.

100. The method of claim 95, wherein the leukocyte comprises a T-cell, a B-cell, an NK cell, or a macrophage.

101. A method of evaluating a potential therapeutic agent, the method comprising:
a) contacting a biological sample with a potential therapeutic agent, wherein the potential therapeutic agent comprises a leukocyte,
b) measuring a metabolic shift in the biological sample from a baseline metabolic signature for the biological sample; and
c) classifying cell state within the biological sample based upon the metabolic shift measured.

102. The method of claim 101, wherein the baseline metabolic signature is determined by imaging the biological sample with fluorescence lifetime imaging microscopy (FLIM).

103. The method of claim 102, wherein FLIM comprises multi-channel FLIM.

104. The method of claim 103, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

105. The method of claim 104, wherein the signal from the first intrinsic fluorophore and the signal from the second intrinsic fluorophore are identified by phasor segmentation analysis.

106. The method of claim 104, wherein the baseline metabolic signature is determined by calculating a lifetime metabolic ratio (LMR), wherein the LMR is calculated by dividing a signal from the first intrinsic fluorophore corresponding to the amount of enzyme-bound NAD by a signal from the second intrinsic fluorophore corresponding to the amount of enzyme-bound FAD.

107. The method of claim 101, wherein the metabolic shift is measured by FLIM.

108. The method of claim 107, wherein the metabolic shift is measured by multi-channel FLIM.

109. The method of claim 108, wherein multi-channel FLIM provides a signal from a first intrinsic fluorophore and a signal from a second intrinsic fluorophore, wherein the first intrinsic fluorophore is NAD(P)H and the second intrinsic fluorophore is FAD.

110. The method of claim 109, wherein the metabolic shift is determined by calculating a lifetime metabolic ratio (LMR), wherein the LMR is calculated by dividing a signal from the first intrinsic fluorophore corresponding to the amount of enzyme-bound NAD by a signal from the second intrinsic fluorophore corresponding to the amount of enzyme-bound FAD.

111. The method of claim 110, wherein the signal from the first intrinsic fluorophore and the signal from the second intrinsic fluorophore are identified by phasor segmentation analysis.

112. The method of claim 101, wherein a metabolic shift above a threshold value is indicative of cell death.

113. The method of claim 101, wherein a metabolic shift within a first range of reference values is indicative of apoptosis, and/or wherein a metabolic shift within a second range of reference values is indicative of necrosis.

114. The method of any one of claim 101, wherein the biological sample comprises a tissue sample.

115. The method of claim 114, wherein the tissue sample comprises a tumor tissue sample.

116. A method of evaluating a biological sample, comprising
a) imaging the biological sample with OCM/dOCM to identify a live cell fraction in the biological sample; and
b) imaging the identified live cell fraction with FLIM to classify the cell state within the identified live cell fraction.

117. The method of claim 116, further comprising:
c) contacting the identified and classified live cell fraction with one or more potential therapeutic agents; and
d) imaging the identified live cell fraction with FLIM to re-classify the cell state within the identified live cell fraction to measure classification differences pre- and post-contacting of the identified and classified live cell fraction with the one or more potential therapeutic agents.

118. An ex vivo method of assessing one or more potential therapeutic agents, comprising
a) imaging a biological sample with OCM and/or dOCM to identify a live cell fraction in the biological sample;
b) imaging the identified live cell fraction with FLIM to classify the cell state within the identified live cell fraction;
c) contacting the identified and classified live cell fraction with one or more potential therapeutic agents; and
d) imaging the identified live cell fraction with OCM/dOCM and/or FLIM to re-classify the cell state within the identified live cell fraction to measure classification differences pre- and post-contacting of the identified and classified live cell fraction with the one or more potential therapeutic agents.

119. An ex vivo method of assessing a biological sample, comprising:
providing
a biological sample, and
a processor comprising software that, when executed, causes the processor to manually or automatically:
image the biological sample with OCM and/or dOCM to identify a live cell fraction in the biological sample; and
image the identified live cell fraction with FLIM to classify the cell state within the identified live cell fraction; and
provide a report to a user of the identified and classified live cell fraction;
executing the processor to
image the biological sample with OCM and/or dOCM to identify a live cell fraction in the biological sample; and
image the identified live cell fraction with OCM/dOCM and/or FLIM to classify the cell state within the identified live cell fraction, and
provide a report to a user of the identified and classified live cell fraction.

120. An ex vivo method of assessing one or more potential therapeutic agents, comprising providing:
a biological sample, and
a processor comprising software that, when executed, causes the processor to manually or automatically:
image the biological sample with OCM and/or dOCM to identify a live cell fraction in the biological sample;
image the identified live cell fraction with FLIM to classify the cell state within the identified live cell fraction;
contact an identified and classified live cell fraction with one or more potential therapeutic agents after an identification and classification of a live cell fraction with OCM and/or dOCM and FLIM;
image the identified and classified live cell fraction with FLIM to re-classify the cell state within the identified and classified live cell fraction;
measure classification differences pre- and post-contacting of identified and classified live cell fraction with the one or more potential therapeutic agents;
provide a report to a user of one or more of:
an identified and classified live cell fraction;
a re-classified identified and classified live cell fraction; and
classification differences pre- and post-contacting of identified and classified live cell fraction with the one or more potential therapeutic agents;
executing the software to:
image the biological sample with optical coherence microscopy (OCM) and/or dynamic optical coherence microscopy (dOCM) to identify a live cell fraction in the biological sample;
image the identified live cell fraction with FLIM to classify the cell state within the identified live cell fraction;
contact an identified and classified live cell fraction with one or more potential therapeutic agents after an identification and classification of a live cell fraction with OCM and/or dOCM and FLIM;
image the identified and classified live cell fraction with fluorescence lifetime imaging microscopy (FLIM) to re-classify the cell state within the identified and classified live cell fraction;
measure classification differences pre- and post-contacting of identified and classified live cell fraction with the one or more potential therapeutic agents;
provide a report to a user of one or more of:
an identified and classified live cell fraction;
a re-classified identified and classified live cell fraction; and
classification differences pre- and post-contacting of identified and classified live cell fraction with the one or more potential therapeutic agents.

121. A system comprising:
a biological sample, and
a processor comprising software that, when executed, causes the processor to manually or automatically:
image the biological sample with OCM/dOCM to identify a live cell fraction in the biological sample; and
image the identified live cell fraction with FLIM to classify the cell state within the identified live cell fraction; and
provide a report to a user of the identified and classified live cell fraction.

122. A system comprising:
a biological sample, and
a processor comprising software that, when executed, causes the processor to manually or automatically:
image the biological sample with OCM/dOCM to identify a live cell fraction in the biological sample;
image the identified live cell fraction with FLIM to classify the cell state within the identified live cell fraction;
contact an identified and classified live cell fraction with one or more potential therapeutic agents after an identification and classification of a live cell fraction with OCM and/or dOCM and FLIM;
re-image the identified and classified live cell fraction with OCM/dOCM and/or FLIM to re-classify the cell state within the identified and classified live cell fraction;
measure classification differences pre- and post-contacting of identified and classified live cell fraction with the one or more potential therapeutic agents;
provide a report to a user of one or more of:
an identified and classified live cell fraction;
a re-classified identified and classified live cell fraction; and
classification differences pre- and post-contacting of identified and classified live cell fraction with the one or more potential therapeutic agents.
